(19) United States
(12) Patent Application Publication
(10) Pub. No.: US 2023/0074615 A1
(43) Pub. Date: Mar. 9, 2023

(54) MMP-9 ANTIBODIES AND METHODS OF USE THEREOF

(71) Applicants: THE REGENTS OF THE UNIVERSITY OF CALIFORNIA, Oakland, CA (US); DUKE UNIVERSITY, Durham, NC (US)

(72) Inventors: Xin GE, Riverside, CA (US); Tyler LOPEZ, Riverside, CA (US); Ru-Rong JI, Durham, NC (US)

(73) Assignees: THE REGENTS OF THE UNIVERSITY OF CALIFORNIA, Oakland, CA (US); DUKE UNIVERSITY, Durham, NC (US)

(21) Appl. No.: 17/612,514

(22) PCT Filed: May 21, 2020
(86) PCT No.: PCT/US20/34076

§ 371 Date: Nov. 18, 2021

Related U.S. Application Data
(60) Provisional application No. 62/851,001, filed on May 21, 2019.

(51) Int. Cl. C07K 16/40; A61P 23/00

(52) U.S. Cl. CPC C07K 16/40; A61P 23/00; A61K 2039/505

(57) ABSTRACT

Certain embodiments provide an isolated anti-matrix metalloproteinase-9 (MMP-9) antibody or fragment thereof, as well as methods of use thereof.

### RELATED APPLICATION

This application claims the benefit of priority of U.S. Provisional Application Ser. No. 62/851,001 filed on May 21, 2019, which application is incorporated by reference herein.

### GOVERNMENT FUNDING

This invention was made with government support under GM115672 and DE017794 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Sep. 14, 2022, is named 12111_012US1_SL.txt and is 21,254 bytes in size.

### BACKGROUND OF THE INVENTION

Accounting for ˜1% of the human genome, extracellular proteases are important signaling molecules that exist in a delicate balance to maintain systematic homeostasis (Turk B, et al., EMBO J. 31, 1630-1643 (2012); Deu, et al., Nat Struct Mol Biol. 19, 9-16 (2012)). Dysregulation of proteolysis causes a variety of disorders ranging from cancer, inflammation, and osteoporosis to neuropathic pain and degenerative diseases (López-Otin C1, Matrisian L M. Nat Rev Cancer. 7, 800-808 (2007); Prassas, et al., Nat Rev Drug Discov. 14, 183-202 (2015); Troeberg et al., Biochim Biophys Acta. 1824, 133-145 (2012); Ji et al., Trends Pharmacol Sci. 30, 336-340 (2009); De Strooper B. Physiol Rev. 90, 465-494 (2010)). Conventional drug discovery strategies led to protease inhibitors currently on the market targeting a small fraction of therapeutically relevant proteases (Drag M, Salvesen G S. Nat Rev Drug Discov. 9, 690-701 (2010); Turk B. Nat Rev Drug Discov. 5, 785-799 (2006)). Small molecule inhibitors are often limited by lack of specificity and/or appropriate pharmacokinetic properties required for a successful protease inhibition therapy (Overall C M, Kleifeld O. Br J Cancer. 94, 941-946 (2006); Vandenbroucke R, Libert C. Nat Rev Drug Discov. 13, :904-927 (2014); Vassar R. Alzheimers Res Ther. 6, 89 (2014)). Conversely, biologics, e.g. monoclonal antibodies (mAbs) provide exquisite specificity capable of distinguishing between closely related protease family members (Wu, et al., Proc Natl Acad Sci USA. 104, 19784-19789 (2007); Devy et al., Cancer Res. 69, 1517-1526 (2009); Atwal, et al., Sci Transl Med. 3, 84ra43 (2011); Schneider et al., J Mol Biol. 415, 699-715 (2012); Kenniston et al., J Biol Chem. 289, 23596-608 (2014); David et al., Sci Transl Med. 8, 353ra112 (2016); Nam et al., Proc Natl Acad Sci USA 113, 14970-14975 (2016)). Their stability in serum, potential to cross blood-brain barrier, novel design as prodrugs, and improved effector functions offer significant advantages over the small-molecule approach (Yu et al., Sci Transl Med. 6, 261ra154 (2014); Sharma S K, Bagshawe K D. Adv Drug Deliv Rev. 118, 2-7 (2017); Wang et al., Protein Cell. 9, 63-73 (2018)). However, current mAb discovery technologies such as hybridoma, phage panning, and cell surface display coupled flow cytometry, all rely on affinity-based selection/screening. Consequently, valuable inhibitory clones tend to be lost during the process, and there is a high level of probability that very few, or even none, of the isolated binders are inhibitory. Furthermore, generated mAbs often exhibit suboptimal inhibition properties and are vulnerable to be cleaved by the protease target. Thus, new methods for the rapid and effective identification of protease inhibitory antibodies are needed. Additionally, there is a need for new protease inhibitory antibodies, in particular, antibodies that inhibit matrix metalloproteinase-9 (MMP-9).

### SUMMARY OF THE INVENTION

Certain embodiments provide an isolated anti-matrix metalloproteinase-9 (MMP-9) antibody or fragment thereof, comprising one or more complementarity determining regions (CDRs) selected from the group consisting of:

(a) a light chain CDR1 having at least 80% sequence identity to an amino acid sequence of RASQSVSSAVA (SEQ ID NO:2);

(b) a light chain CDR2 having at least 80% sequence identity to an amino acid sequence of SASSLYS (SEQ ID NO:3);

(c) a light chain CDR3 having at least 80% sequence identity to an amino acid sequence of any one of QQSYHPLFT (SEQ ID NO:4), QQASHLIT (SEQ ID NO:12), QQYAALIT (SEQ ID NO:20) and QQGGGASLIT (SEQ ID NO:38);

(d) a heavy chain CDR1 having at least 80% sequence identity to an amino acid sequence of any one of GFNIYSYSIH (SEQ ID NO:6), GFNIYSSSMH (SEQ ID NO:15) and GFNISSSYIH (SEQ ID NO:23);

(e) a heavy chain CDR2 having at least 80% sequence identity to an amino acid sequence of any one of YIYPSSGYTYYADSVK (SEQ ID NO:7), YIYSSYGYTYYADSVK (SEQ ID NO:16), SISSSSGYTSYADSVK (SEQ ID NO:24) and SIYSYYGYTYYADSVK (SEQ ID NO:27); and

(f) a heavy chain CDR3 having at least 80% sequence identity to an amino acid sequence of any one of SSLAWAQDRVYKPVEAMTWAYGMDY (SEQ ID NO:8), RFEPGLLKRNKRWISYTLCEAGYGMDY (SEQ ID NO:17), KYMVFGTRMGWVEHTDFAGQGYYAMDY (SEQ ID NO:25) and CKLYTSYMIPVGSDSVNRCMSSYGMDY (SEQ ID NO:28).

Certain embodiments provide a composition comprising an anti-MMP-9 antibody, or fragment thereof, as described herein and a carrier.

Certain embodiments provide an isolated polynucleotide comprising a nucleotide sequence encoding the isolated anti-MMP-9 antibody, or fragment thereof, as described herein.

Certain embodiments provide a vector comprising the polynucleotide as described herein.

Certain embodiments provide a cell comprising a polynucleotide as described herein or a vector as described herein.

Certain embodiments provide a method of detecting the presence of MMP-9 in a cell, the method comprising contacting the cell with an isolated anti-MMP-9 antibody, or fragment thereof, as described herein and detecting whether a complex is formed between the anti-MMP-9 antibody and MMP-9.

Certain embodiments provide a method of inhibiting the activity of MMP-9, comprising contacting MMP-9 with an isolated anti-MMP-9 antibody, or fragment thereof, as described herein.

Certain embodiments provide a method for treating pain in a mammal, comprising administering an effective amount of an isolated anti-MMP-9 antibody, or fragment thereof, as described herein to the mammal.

Certain embodiments provide an isolated anti-MMP-9 antibody, or fragment thereof, as described herein for the prophylactic or therapeutic treatment of pain.

Certain embodiments provide the use of an isolated anti-MMP-9 antibody, or fragment thereof, as described herein to prepare a medicament for the treatment of pain in a mammal.

Certain embodiments provide a method for treating a stroke in a mammal, comprising administering an effective amount of an isolated anti-MMP-9 antibody, or fragment thereof, as described herein to the mammal.

Certain embodiments provide an isolated anti-MMP-9 antibody, or fragment thereof, as described herein for the prophylactic or therapeutic treatment of a stroke.

Certain embodiments provide the use of an isolated anti-MMP-9 antibody, or fragment thereof, as described herein to prepare a medicament for the treatment of a stroke in a mammal.

Certain embodiments provide an isolated anti-MMP-9 antibody, or fragment thereof, as described herein for use in medical therapy.

Certain embodiments provide a kit comprising an isolated anti-MMP-9 antibody, or fragment thereof, as described herein, packaging material, and instructions for administering the antibody, or a fragment thereof, to a mammal to treat pain.

Certain embodiments provide a kit comprising an isolated anti-MMP-9 antibody, or fragment thereof, as described herein, packaging material, and instructions for administering the antibody, or a fragment thereof, to a mammal to treat a stroke.

The invention also provides processes and intermediates disclosed herein that are useful for preparing antibodies, or fragments thereof, and compositions described herein.

### BRIEF DESCRIPTION OF THE FIGURES

FIGS. 1A-1B. Functional selection for protease inhibitory antibodies. (FIG. 1A) Scheme showing that three recombinant proteins are simultaneously co-expressed in the periplasmic space of E. coli—a clone from the Fab library, the protease of interest, and the modified β-lactamase TEM-1 with a cleavable peptide insertion. The protease extracellular/catalytic domain under a lac promoter and TEM-1 under its native promoter are cloned into a low copy number (p15A ori) plasmid of chloramphenicol resistance (CmR). The antibody Fab library under a phoA promoter is cloned into a medium copy number (pBR322 ori) plasmid carrying kanamycin resistance (KanR). If the Fab has no inhibition, the protease will cleave TEM-1 leading to cell death in the presence of ampicillin. An inhibitory Fab blocks proteolytic activity which allows TEM-1 to remain intact, resulting in cell growth on ampicillin plates. (FIG. 1B) Selection windows for cdMMP-9. TEM-1 was modified by inserting the protease specific cleavage peptide sequences (shown in parentheses) between Gly196 and Glu197 of TEM-1 (FIG. 8). Survival curves of E. coli cells transformed with modified TEM-1 were measured (solid), and compared to those for cells also co-expressing the associated proteases (dashed). Survival curve of wt TEM-1 is also shown. The experiments were repeated three times. FIG. 1B discloses “RLPLGI” as SEQ ID NO: 31.

FIG. 2. Inhibition potency and selectivity for anti-MMP9 IgG L13. Inhibition assays were measured using FRET peptide substrates.

FIG. 3. In vitro proteolytic stability. SDS-PAGE of 1 μM Fabs after incubation with 1 μM of the target protease for 12 hours. Densitometric analysis was performed to determine the relative amounts of remained Fabs.

FIG. 4. Inhibitory functions of isolated mAbs on proteolysis of physiological/macromolecular substrates. Fab L13 blocked MMP-9 from hydrolyzing type I collagen. HEK293F cell cultures expressing APP₅₇₁₋₆₉₆ were incubated with IgG for 72 hours. Generated Aβ₄₀ was measured by ELISA.

FIGS. 5A-5B. Inhibition mechanisms of anti-MMP9 Fabs. (FIG. 5A) Lineweaver-Burke plots of cdMMP-9 in the presence of 62.5, 250, 500 nM Fab L13 (left) or 66, 33, 16.5 nM Fab H4 (right). Unaltered Vₘₐₓ and increased Kₘ with increasing Fab concentrations indicated that L13 was a competitive inhibitor. In contrast, increasing Vₘₐₓ and increased Kₘ with increasing Fab concentrations indicate that H4 was an uncompetitive inhibitor. (FIG. 5B) Competitive ELISA of Fab L13 (left) or Fab H4 (right) on immobilized cdMMP-9 in the presence of 1 nM-125 nM nTIMP-2. Results suggested that L13 had its epitope overlapping with that nTIM-2, while the epitope of H4 did not overlap with nTIMP-2. Collectively, L13 was an active-site competitive inhibitor and H4 was an allosteric uncompetitive inhibitor.

FIGS. 6A-6B. Analgesic effects of MMP-9 inhibitor IgG L13 in neuropathic pain induced by the chemotherapy agent paclitaxel (PTX) in mice. 200 ng IgG L13 was I.V. administered on day 15 after PTX injections. Behavior tests of neuropathic pain symptom mechanical allodynia, evaluated by paw withdrawal threshold (FIG. 6A) and frequency (FIG. 6B) were performed in a blinded manner (n=7 mice for control IgG, and n=6 for L13 IgG). ***p<0.001, two-way ANOVA with Turkey's posthoc test.

FIG. 7. Periplasmic production of extracellular/catalytic domains of human/fungal proteases in their active soluble format. Purified proteases were analyzed by SDS-PAGE, and their enzymatic kinetics were measured with FRET peptide substrates.

FIG. 8. Design of β-lactamase TEM1 sensor for protease inhibition. Structure (PBD, 4ZJ3) showing that the location of cleavage peptide insertion (between Gly196 and Glu197) is on a loop far away from the active site where substrates, e.g. cephalexin, bind. Images were generated by using PyMol (PDB=4ZJ2).

FIG. 9. Binding kinetics of isolated Fabs to protease targets. kₒₙ and k_{off} values were measured by bio-layer interferometry and used for K_{D} calculation. Only Fab clones with K₁<500 nM are shown in the same order as in Table 1.

FIG. 10. Inhibition potencies of isolated Fabs. Inhibition IC₅₀s were measured by using FRET peptide substrates. Only Fab clones with K₁<500 nM are shown in the same order as in Table 1. Inhibition selectivity of representative Fabs was also tested. For anti-cdMMP9 clone H3, its IgG instead of Fab is shown.

FIG. 11. Active sites and electrostatic surface potentials of representative proteases. Side chains of the catalytic residues are highlighted. Surface topologies display the reactive clefts or cavities of diverse conformation. Images were generated with indicated PDB files by using PyMol.

### DETAILED DESCRIPTION

Proteases represent one of the largest families of pharmaceutical targets. To inhibit pathogenic proteases with desired selectivity, monoclonal antibodies (mAbs) hold a great promise as research tools and therapeutic agents. However, identification of mAbs with inhibitory functions is challenging because current antibody discovery methods rely on binding rather than inhibition. This study developed a highly efficient selection method for protease inhibitory mAbs by co-expressing three recombinant proteins in the periplasmic space of Escherichia coli—an antibody clone, a protease of interest, and a β-lactamase modified by insertion of a protease cleavable peptide sequence. During functional selection, inhibitory antibodies prevent the protease from cleaving the modified β-lactamase thereby allowing the cell to survive in the presence of ampicillin. Using this method to select from synthetic human antibody libraries, we isolated a panel of mAbs that inhibit MMP-9. Notably, a large percentage of the identified binders were inhibitory. Isolated mAb inhibitors exhibited nanomolar potency, exclusive selectivity, excellent proteolytic stability, and desired biological functions. For example, IgG L13 inhibited MMP-9 but not MMP-2/-12/-14 and significantly relieved neuropathic pain development in mice.

### Anti-MMP9 Antibodies or Fragments Thereof

Accordingly, certain embodiments provide antibodies and antigen-binding portions of antibodies that specifically bind to MMP-9 (i.e., an anti-MMP antibody, or fragment thereof).

In some embodiments, an anti-MMP-9 antibody, or fragment thereof, comprises: (1) one or more complementarity determining region (CDR) sequences; (2) a heavy chain variable region sequence; and/or (3) a light chain variable region sequence, as described herein (e.g., as described in Table 3 below).

In certain embodiments, an isolated anti-MMP-9 antibody or fragment thereof, comprises one or more CDRs selected from the group consisting of:

(a) a light chain CDR1 having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of RASQSVSSAVA (SEQ ID NO:2);

(b) a light chain CDR2 having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of SASSLYS (SEQ ID NO:3);

(c) a light chain CDR3 having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one of QQSYHPLFT (SEQ ID NO:4), QQASHLIT (SEQ ID NO:12) and QQYAALIT (SEQ ID NO:20);

(d) a heavy chain CDR1 having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one of GFNIYSYSIH (SEQ ID NO:6), GFNIYSSSMH (SEQ ID NO:15) and GFNISSSYIH (SEQ ID NO:23);

(e) a heavy chain CDR2 having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one of YIYPSSGYTYYADSVK (SEQ ID NO:7), YIYSSYGYTYYADSVK (SEQ ID NO:16), SISSSSGYTSYADSVK (SEQ ID NO:24) and SIYSYYGYTYYADSVK (SEQ ID NO:27); and

(f) a heavy chain CDR3 having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one of SSLAWAQDRVYKPVEAMTWAYGMDY (SEQ ID NO:8), RFEPGLLKRNKRWISYTLCEAGYGMDY (SEQ ID NO:17), KYMVFGTRMGWVEHTDFAGQGYYAMDY (SEQ ID NO:25) and CKLYTSYMIPVGSDSVNRCMSSYGMDY (SEQ ID NO:28).

In certain embodiments, an isolated anti-MMP-9 antibody or fragment thereof, comprises one or more CDRs selected from the group consisting of:

(a) a light chain CDR1 having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one of RASQSVSSAVA (SEQ ID NO:2);

(b) a light chain CDR2 having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of SASSLYS (SEQ ID NO:3);

(c) a light chain CDR3 having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one of QQSYHPLFT (SEQ ID NO:4), QQASHLIT (SEQ ID NO:12), QQYAALIT (SEQ ID NO:20) and QQGGGASLIT (SEQ ID NO:38);

(d) a heavy chain CDR1 having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one of GFNIYSYSIH (SEQ ID NO:6), GFNIYSSSMH (SEQ ID NO:15) and GFNISSSYIH (SEQ ID NO:23);

(e) a heavy chain CDR2 having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one of YIYPSSGYTYYADSVK (SEQ ID NO:7), YIYSSYGYTYYADSVK (SEQ ID NO:16), SISSSSGYTSYADSVK (SEQ ID NO:24) and SIYSYYGYTYYADSVK (SEQ ID NO:27); and

(f) a heavy chain CDR3 having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one of SSLAWAQDRVYKPVEAMTWAYGMDY (SEQ ID NO:8), RFEPGLLKRNKRWISYTLCEAGYGMDY (SEQ ID NO:17), KYMVFGTRMGWVEHTDFAGQGYYAMDY (SEQ ID NO:25) and CKLYTSYMIPVGSDSVNRCMSSYGMDY (SEQ ID NO:28).

In some embodiments, an anti-MMP-9 antibody, or fragment thereof, comprises two, three, four, five or six CDRs as described above (e.g., each CDR is selected from one of (a)-(f)).

In certain embodiments, the anti-MMP-9 antibody, or fragment thereof, as described herein comprises one or more CDRs selected from the group consisting of:

(a) a light chain CDR1 comprising the amino acid sequence of SEQ ID NO:2;

(b) a light chain CDR2 comprising the amino acid sequence of SEQ ID NO:3;

(c) a light chain CDR3 comprising the amino acid sequence of any one of SEQ ID NOs:4, 12, 20 and 38;

(d) a heavy chain CDR1 comprising the amino acid sequence of any one of SEQ ID NOs:6, 15 and 23;

(e) a heavy chain CDR2 comprising the amino acid sequence of any one of SEQ ID NOs:7, 16, 24 and 27; and

(f) a heavy chain CDR3 comprising the amino acid sequence of any one of SEQ ID NOs:8, 17, 25 and 28.

In some embodiments, an anti-MMP-9 antibody, or fragment thereof, comprises two, three, four, five or six CDRs as described above (e.g., each CDR is selected from one of (a)-(f)).

For example, in certain embodiments, the anti-MMP-9 antibody, or fragment thereof, as described herein comprises:

(a) a light chain CDR1 comprising the amino acid sequence of SEQ ID NO:2;

(b) a light chain CDR2 comprising the amino acid sequence of SEQ ID NO:3;

(c) a light chain CDR3 comprising the amino acid sequence of any one of SEQ ID NOs:4, 12, 20 and 38;

(d) a heavy chain CDR1 comprising the amino acid sequence of any one of SEQ ID NOs:6, 15 and 23;

(e) a heavy chain CDR2 comprising the amino acid sequence of any one of SEQ ID NOs:7, 16, 24 and 27; and

(f) a heavy chain CDR3 comprising the amino acid sequence of any one of SEQ ID NOs:8, 17, 25 and 28.

In some embodiments, an anti-MMP-9 antibody, or a fragment thereof, comprises a light chain sequence, or a fragment thereof, and/or a heavy chain sequence, or a fragment thereof, derived from any of the following antibodies described herein: H3, H4, L13 and H25. The amino acid sequences of the light chain variable domain (VL) and heavy chain variable domain (VH) of these anti-MMP-9 antibody clones are set forth in Table 3 below.

In certain embodiments, an anti-MMP-9 antibody, or a fragment thereof, comprises a VL as in any of the embodiments provided herein, and/or a VH as in any of the embodiments provided herein.

In certain embodiments, an anti-MMP-9 antibody described herein, or fragment thereof, comprises a light chain variable region comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of:

(a)
(SEQ ID NO: 1)
DIQMTQSPSSLSASVGDRVTITCRASQSVSSAVAWYQQKPGKAPKLLIYS
ASSLYSGVPSRFSGSRSGTDFTLTISSLQPEDFATYYCQQSYHPLFTFGQ
GTKVEIKR;
(b)
(SEQ ID NO: 9)
DIQMTQSPSSLSASVGDRVTITCRASQSVSSAVAWYQQKPGKAPKLLIYS
ASSLYSGVPSRFSGSRSGTDFTLTISSLQPEDFATYYCQQASHLITFGQG
TKVEIKR;
and
(c)
(SEQ ID NO: 18)
DIQMTQSPSSLSASVGDRVTITCRASQSVSSAVAWYQQKPGKAPKLLIYS
ASSLYSGVPSRFSGSRSGTDFTLTISSLQPEDFATYYCQQYAALITFGQG
TKVEIKR.

In certain embodiments, an anti-MMP-9 antibody described herein, or fragment thereof, comprises a light chain variable region comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of:

(a)
(SEQ ID NO: 1)
DIQMTQSPSSLSASVGDRVTITCRASQSVSSAVAWYQQKPGKAPKLLIYS
ASSLYSGVPSRFSGSRSGTDFTLTISSLQPEDFATYYCQQSYHPLFTFGQ
GTKVEIKR;
(b)
(SEQ ID NO: 9)
DIQMTQSPSSLSASVGDRVTITCRASQSVSSAVAWYQQKPGKAPKLLIYS
ASSLYSGVPSRFSGSRSGTDFTLTISSLQPEDFATYYCQQASHLITFGQG
TKVEIKR;
(c)
(SEQ ID NO: 18)
DIQMTQSPSSLSASVGDRVTITCRASQSVSSAVAWYQQKPGKAPKLLIYS
ASSLYSGVPSRFSGSRSGTDFTLTISSLQPEDFATYYCQQYAALITFGQG
TKVEIKR;
and
(d)
(SEQ ID NO: 37)
DIQMTQSPSSLSASVGDRVTITCRASQSVSSAVAWYQQKPGKAPKLLIYS
ASSLYSGVPSRFSGSRSGTDFTLTISSLQPEDFATYYCQQGGGASLITFG
QGTKVEIKR.

In some embodiments, an anti-MMP-9 antibody, or fragment thereof, comprises a light chain variable region comprising the amino acid sequence of any one of SEQ ID NOs:1, 9, 18 and 37. In some embodiments, an anti-MMP-9 antibody, or fragment thereof, comprises a light chain variable region consisting of the amino acid sequence of any one of SEQ ID NOs:1, 9, 18 and 37.

In certain embodiments, an isolated anti-MMP-9 antibody described herein, or fragment thereof, comprises a heavy chain variable region comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of:

(a)
(SEQ ID NO: 5)
EVQLVESGGGLVQPGGSLRLSCAASGFNIYSYSIHWVRQAPGKGLEWVAY
IYPSSGYTYYADSVKGRFTISADTSKNTAYLQMNSLRAEDTAVYYCARSS
LAWAQDRVYKPVEAMTWAYGMDYWGQGTLVTVSSAS;
(b)
(SEQ ID NO: 13)
EVQLVESGGGLVQPGGSLRLSCAASGFNIYSSSMHWVRQAPGKGLEWVAY
IYSSYGYTYYADSVKGRFTISADTSKNTAYLQMNSLRAEDTAVYYCARRF
EPGLLKRNKRWISYTLCEAGYGMDYWGQGTLVTVSSAS;
(c)
(SEQ ID NO: 21)
EVQLVESGGGLVQPGGSLRLSCAASGFNISSSYIHWVRQAPGKGLEWVAS
ISSSSGYTSYADSVKGRFTISADTSKNTAYLQMNSLRAEDTAVYYCARKY
MVFGTRMGWVEHTDFAGQGYYAMDYWGQGTLVTVSSAS;
and
(d)
(SEQ ID NO: 26)
EVQLVESGGGLVQPGGSLRLSCAASGFNIYSSSMHWVRQAPGKGLEWVAS
IYSYYGYTYYADSVKGRFTISADTSKNTAYLQMNSLRAEDTAVYYCARCK
LYTSYMIPVGSDSVNRCMSSYGMDYWGQGTLVTVSSAS.

In some embodiments, an anti-MMP-9 antibody, or fragment thereof, comprises a heavy chain variable region comprising the amino acid sequence of any one of SEQ ID NOs:5, 13, 21 and 26. In some embodiments, an anti-MMP-9 antibody, or fragment thereof, comprises a heavy chain variable region consisting of the amino acid sequence of any one of SEQ ID NOs:5, 13, 21 and 26.

In some embodiments, an anti-MMP-9 antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of SEQ ID NOs: 1, 9 or 18 and further comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of SEQ ID NOs:5, 13, 21 or 26.

In some embodiments, an anti-MMP-9 antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of SEQ ID NOs: 1, 9, 18 or 37 and further comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of SEQ ID NOs:5, 13, 21 or 26.

In some embodiments, an anti-MMP-9 antibody, or fragment thereof, comprises a light chain variable region consisting of an amino acid sequence of SEQ ID NOs: 1, 9 or 18 and further comprises a heavy chain variable region consisting of an amino acid sequence of SEQ ID NOs:5, 13, 21 or 26.

In some embodiments, an anti-MMP-9 antibody, or fragment thereof, comprises a light chain variable region consisting of an amino acid sequence of SEQ ID NOs: 1, 9, 18 or 37 and further comprises a heavy chain variable region consisting of an amino acid sequence of SEQ ID NOs:5, 13, 21 or 26.

### Clone H4

In some embodiments, an anti-MMP-9 antibody, or fragment thereof, comprises a light chain CDR1 comprising the amino acid sequence of SEQ ID NO:2, a light chain CDR2 comprising the amino acid sequence of SEQ ID NO:3, and a light chain CDR3 comprising the amino acid sequence of SEQ ID NO:4. In some embodiments, an anti-MMP-9 antibody, or fragment thereof, comprises a heavy chain CDR1 comprising the amino acid sequence of SEQ ID NO:6, a heavy chain CDR2 comprising the amino acid sequence of SEQ ID NO:7, and a heavy chain CDR3 comprising the amino acid sequence of SEQ ID NO:8. In some embodiments, an anti-MMP-9 antibody, or fragment thereof, comprises a light chain CDR1-3 and a heavy chain CDR1-3 comprising the amino acid sequences of SEQ ID NOs:2, 3, 4, 6, 7 and 8, respectively. In some embodiments, an anti-MMP-9 antibody, or fragment thereof, comprises a light chain CDR1-3 and a heavy chain CDR1-3 consisting of the amino acid sequences of SEQ ID NOs:2, 3, 4, 6, 7 and 8, respectively.

In some embodiments, an anti-MMP-9 antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:1. In some embodiments, an anti-MMP-9 antibody, or fragment thereof, comprises a light chain variable region comprising the amino acid sequence of SEQ ID NO:1. In some embodiments, an anti-MMP-9 antibody, or fragment thereof, comprises a light chain variable region consisting of the amino acid sequence of SEQ ID NO:1.

In some embodiments, an anti-MMP-9 antibody, or fragment thereof, comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:5. In some embodiments, an anti-MMP-9 antibody, or fragment thereof, comprises a heavy chain variable region comprising the amino acid sequence of SEQ ID NO:5. In some embodiments, an anti-MMP-9 antibody, or fragment thereof, comprises a heavy chain variable region consisting of the amino acid sequence of SEQ ID NO:5.

In some embodiments, an anti-MMP-9 antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:1 and further comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:5. In some embodiments, an anti-MMP-9 antibody, or fragment thereof, comprises a light chain variable region comprising the amino acid sequence of SEQ ID NO:1 and further comprises a heavy chain variable region comprising the amino acid sequence of SEQ ID NO:5. In some embodiments, an anti-MMP-9 antibody, or fragment thereof, comprises a light chain variable region consisting of the amino acid sequence of SEQ ID NO:1 and further comprises a heavy chain variable region consisting of the amino acid sequence of SEQ ID NO:5.

### Clone H3

In some embodiments, an anti-MMP-9 antibody, or fragment thereof, comprises a light chain CDR1 comprising the amino acid sequence of SEQ ID NO:2, a light chain CDR2 comprising the amino acid sequence of SEQ ID NO:3, and a light chain CDR3 comprising the amino acid sequence of SEQ ID NO:12. In some embodiments, an anti-MMP-9 antibody, or fragment thereof, comprises a heavy chain CDR1 comprising the amino acid sequence of SEQ ID NO:15, a heavy chain CDR2 comprising the amino acid sequence of SEQ ID NO:16, and a heavy chain CDR3 comprising the amino acid sequence of SEQ ID NO:17. In some embodiments, an anti-MMP-9 antibody, or fragment thereof, comprises a light chain CDR1-3 and a heavy chain CDR1-3 comprising the amino acid sequences of SEQ ID NOs:2, 3, 12, 15, 16 and 17, respectively. In some embodiments, an anti-MMP-9 antibody, or fragment thereof, comprises a light chain CDR1-3 and a heavy chain CDR1-3 consisting of the amino acid sequences of SEQ ID NOs:2, 3, 12, 15, 16 and 17, respectively.

In some embodiments, an anti-MMP-9 antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:9. In some embodiments, an anti-MMP-9 antibody, or fragment thereof, comprises a light chain variable region comprising the amino acid sequence of SEQ ID NO:9. In some embodiments, an anti-MMP-9 antibody, or fragment thereof, comprises a light chain variable region consisting of the amino acid sequence of SEQ ID NO:9.

In some embodiments, an anti-MMP-9 antibody, or fragment thereof, comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:13. In some embodiments, an anti-MMP-9 antibody, or fragment thereof, comprises a heavy chain variable region comprising the amino acid sequence of SEQ ID NO:13. In some embodiments, an anti-MMP-9 antibody, or fragment thereof, comprises a heavy chain variable region consisting of the amino acid sequence of SEQ ID NO:13.

In some embodiments, an anti-MMP-9 antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:9 and further comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:13. In some embodiments, an anti-MMP-9 antibody, or fragment thereof, comprises a light chain variable region comprising the amino acid sequence of SEQ ID NO:9 and further comprises a heavy chain variable region comprising the amino acid sequence of SEQ ID NO:13. In some embodiments, an anti-MMP-9 antibody, or fragment thereof, comprises a light chain variable region consisting of the amino acid sequence of SEQ ID NO:9 and further comprises a heavy chain variable region consisting of the amino acid sequence of SEQ ID NO:13.

### Clone L13

In some embodiments, an anti-MMP-9 antibody, or fragment thereof, comprises a light chain CDR1 comprising the amino acid sequence of SEQ ID NO:2, a light chain CDR2 comprising the amino acid sequence of SEQ ID NO:3, and a light chain CDR3 comprising the amino acid sequence of SEQ ID NO:20. In some embodiments, an anti-MMP-9 antibody, or fragment thereof, comprises a heavy chain CDR1 comprising the amino acid sequence of SEQ ID NO:23, a heavy chain CDR2 comprising the amino acid sequence of SEQ ID NO:24, and a heavy chain CDR3 comprising the amino acid sequence of SEQ ID NO:25. In some embodiments, an anti-MMP-9 antibody, or fragment thereof, comprises a light chain CDR1-3 and a heavy chain CDR1-3 comprising the amino acid sequences of SEQ ID NOs:2, 3, 20, 23, 24 and 25, respectively. In some embodiments, an anti-MMP-9 antibody, or fragment thereof, comprises a light chain CDR1-3 and a heavy chain CDR1-3 consisting of the amino acid sequences of SEQ ID NOs: 2, 3, 20, 23, 24 and 25, respectively.

In some embodiments, an anti-MMP-9 antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:18. In some embodiments, an anti-MMP-9 antibody, or fragment thereof, comprises a light chain variable region comprising the amino acid sequence of SEQ ID NO:18. In some embodiments, an anti-MMP-9 antibody, or fragment thereof, comprises a light chain variable region consisting of the amino acid sequence of SEQ ID NO:18.

In some embodiments, an anti-MMP-9 antibody, or fragment thereof, comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:21. In some embodiments, an anti-MMP-9 antibody, or fragment thereof, comprises a heavy chain variable region comprising the amino acid sequence of SEQ ID NO:21. In some embodiments, an anti-MMP-9 antibody, or fragment thereof, comprises a heavy chain variable region consisting of the amino acid sequence of SEQ ID NO:21.

In some embodiments, an anti-MMP-9 antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 18 and further comprises a heavy chain variable region comprising an amino acid sequence that has at least about 80% (e.g., 81%, 82%, 83%, 84%, 85%, 86%, 870%, 88%, 89%, 90%, 91%, 92%, 93%, 94%, 95%, 96%, 97%, 98%, 99% or 100%) sequence identity to SEQ ID NO:21. In some embodiments, an anti-MMP-9 antibody, or fragment thereof, comprises a light chain variable region comprising the amino acid sequence of SEQ ID NO:18 and further comprises a heavy chain variable region comprising the amino acid sequence of SEQ ID NO:21. In some embodiments, an anti-MMP-9 antibody, or fragment thereof, comprises a light chain variable region consisting of the amino acid sequence of SEQ ID NO:18 and further comprises a heavy chain variable region consisting of the amino acid sequence of SEQ ID NO:21.

### Clone H25

In some embodiments, an anti-MMP-9 antibody, or fragment thereof, comprises a light chain CDR1 comprising the amino acid sequence of SEQ ID NO:2, a light chain CDR2 comprising the amino acid sequence of SEQ ID NO:3, and a light chain CDR3 comprising the amino acid sequence of SEQ ID NO:38. In some embodiments, an anti-MMP-9 antibody, or fragment thereof, comprises a heavy chain CDR1 comprising the amino acid sequence of SEQ ID NO:15, a heavy chain CDR2 comprising the amino acid sequence of SEQ ID NO:27, and a heavy chain CDR3 comprising the amino acid sequence of SEQ ID NO:28. In some embodiments, an anti-MMP-9 antibody, or fragment thereof, comprises a light chain CDR1-3 and a heavy chain CDR1-3 comprising the amino acid sequences of SEQ ID NOs:2, 3, 38, 15, 27 and 28, respectively. In some embodiments, an anti-MMP-9 antibody, or fragment thereof, comprises a light chain CDR1-3 and a heavy chain CDR1-3 consisting of the amino acid sequences of SEQ ID NOs:2, 3, 38, 15, 27 and 28, respectively.

In some embodiments, an anti-MMP-9 antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:37. In some embodiments, an anti-MMP-9 antibody, or fragment thereof, comprises a light chain variable region comprising the amino acid sequence of SEQ ID NO:37. In some embodiments, an anti-MMP-9 antibody, or fragment thereof, comprises a light chain variable region consisting of the amino acid sequence of SEQ ID NO:37.

In some embodiments, an anti-MMP-9 antibody, or fragment thereof, comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:26. In some embodiments, an anti-MMP-9 antibody, or fragment thereof, comprises a heavy chain variable region comprising the amino acid sequence of SEQ ID NO:26. In some embodiments, an anti-MMP-9 antibody, or fragment thereof, comprises a heavy chain variable region consisting of the amino acid sequence of SEQ ID NO:26.

In some embodiments, an anti-MMP-9 antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:37 and further comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:26. In some embodiments, an anti-MMP-9 antibody, or fragment thereof, comprises a light chain variable region comprising the amino acid sequence of SEQ ID NO:37 and further comprises a heavy chain variable region comprising the amino acid sequence of SEQ ID NO:26. In some embodiments, an anti-MMP-9 antibody, or fragment thereof, comprises a light chain variable region consisting of the amino acid sequence of SEQ ID NO:37 and further comprises a heavy chain variable region consisting of the amino acid sequence of SEQ ID NO:26.

In certain embodiments, an isolated anti-MMP-9 antibody described herein, or fragment thereof, further comprises at least one heavy chain constant region and/or at least one light chain constant region. Thus, in certain embodiments, the light chain variable region is linked (e.g., through a linker or a direct bond, such as a peptide bond) to a light chain constant region. In certain embodiments, the heavy chain variable region is linked to at least one heavy chain constant region (e.g., 1, 2, or 3). In certain embodiments, the heavy and light chains are linked via one or more disulfide bonds.

In certain embodiments, the antibody or fragment thereof is a recombinant antibody or fragment thereof. In certain embodiments, the antibody or fragment thereof is a chimeric antibody or fragment thereof. In certain embodiments, the antibody or fragment thereof is humanized.

In certain embodiments, an antibody of the invention is a monoclonal antibody or a fragment thereof. In some embodiments, the monoclonal antibody, or fragment thereof, recognizes an epitope within human MMP-9.

In certain embodiments, antibody, or fragment thereof, is a fragment. In certain embodiments, the fragment comprises an antigen-binding domain or a variable region. For example, in certain embodiments, the fragment is a fragment antigen-binding (Fab), F(ab′)₂, Fv, single-chain Fv (scFv), CDR (e.g., CDR-H3), diabody (diabodies), linear antibody or a multispecific antibody prepared from an antibody fragment. In certain embodiments, the fragment is a Fab fragment (e.g., a Fab comprising a human antibody scaffold). In other embodiments, the fragment is a CDR-H3.

In another embodiment, the antibody is a substantially full length antibody, e.g., an IgG antibody, or other antibody class or isotype as defined herein.

In certain embodiments, an isolated anti-MMP-9 antibody described herein, or fragment thereof, is an inhibitor of MMP-9.

The term “inhibitor of MMP-9” as used herein refers to an antibody or fragment thereof that is capable of inhibiting the function of MMP-9 (e.g., inhibits enzymatic activity, e.g., inhibits protease cleavage activity). For example, in certain embodiments, the antibody, or fragment thereof, detectably inhibits the biological activity of MMP-9 as measured, e.g., using an assay described herein. In certain embodiments, the antibody, or fragment thereof, inhibits the biological activity of MMP-9 by at least about 5%, at least about 10%, at least about 20%, at least about 30%, at least about 40%, at least about 50%, at least about 60%, at least about 70%, at least about 80%, or at least about 90%. In certain embodiments, the antibody or fragment thereof is a selective inhibitor of MMP-9. For example, an antibody of the invention may be at least 5, at least 10, at least 50, at least 100, at least 500, or at least 1,000 fold selective for MMP-9 over another MMP in a selected assay (e.g., an assay described in the Examples herein).

In certain embodiments, an isolated anti-MMP-9 antibody described herein, or fragment thereof, further comprises a detectable label.

Certain embodiments of the invention provide an antibody or fragment thereof as described herein.

Certain embodiments of the invention provide a method as described herein for making an antibody of the invention or fragment thereof.

Certain embodiments of the invention provide a method as described herein for isolating an antibody of the invention or fragment thereof from an antibody library.

Certain embodiments of the invention provide an antibody or fragment thereof isolated by a method as described herein.

Certain embodiments provide a composition comprising an anti-MMP-9 antibody as described herein, or fragment thereof, and a carrier. In certain embodiments, the composition is a pharmaceutical composition comprising a pharmaceutically acceptable carrier.

Certain embodiments provide a kit comprising an isolated anti-MMP-9 antibody as described herein, or fragment thereof, packaging material, and instructions for administering the antibody, or a fragment thereof, to a mammal to treat pain. In certain embodiments, the pain is chronic pain. In certain embodiments, the pain is neuropathic pain (e.g., associated with diabetes; associated with a viral infection, such as Shingles (Herpes Zoster) or an HIV infection; or associated with a surgery, such as a thoracotomy or amputation). In certain embodiments, the pain is associated with or results from chemotherapy, nerve injury, trigeminal neuralgia, spinal cord injury, stroke, brain trauma, arthritic pain (e.g., osteoarthritis or rheumatoid arthritis), headache or migraine, cancer or surgery (e.g., postoperative pain). In certain embodiments, the kit further comprises at least one additional therapeutic agent. In certain embodiments, the at least one additional therapeutic agent is useful for treating pain. In certain embodiments, the at least one additional therapeutic agent is a steroid, a non-steroid anti-inflammatory drug (NSAIDs), a nerve blocker, an anti-depressant, gabapentin, Lyrica, a local anesthetic (e.g., lidocaine) or an opioid.

Certain embodiments provide a kit comprising an isolated anti-MMP-9 antibody as described herein, or fragment thereof, packaging material, and instructions for administering the antibody, or a fragment thereof, to a mammal to treat a stroke. In certain embodiments, the kit further comprises at least one additional therapeutic agent. In certain embodiments, the at least one additional therapeutic agent is useful for treating a stroke.

As used herein, the term “antibody” includes a single-chain variable fragment (scFv or “nanobody”), humanized, fully human or chimeric antibodies, single-chain antibodies, diabodies, and antigen-binding fragments of antibodies that do not contain the Fc region (e.g., Fab fragments). In certain embodiments, the antibody is a human antibody or a humanized antibody. A “humanized” antibody contains only the three CDRs (complementarity determining regions) and sometimes a few carefully selected “framework” residues (the non-CDR portions of the variable regions) from each donor antibody variable region recombinantly linked onto the corresponding frameworks and constant regions of a human antibody sequence. A “fully humanized antibody” is created in a hybridoma from mice genetically engineered to have only human-derived antibody genes or by selection from a phage-display library of human-derived antibody genes.

A scFv is a fusion protein of the variable region of the heavy (V_{H}) and light chains (V_{L}) of an immunoglobulin that is connected by means of a linker peptide. The linker is usually short, about 10-25 amino acids in length. If flexibility is important, the linker will contain a significant number of glycines. If solubility is important, serines or theonines will be utilized in the linker. The linker may link the amino-terminus of the V_{H} to the carboxy-terminus of the V_{L}, or the linker may link the carboxy-terminus of the V_{H} to the amino-terminus of the V_{L}. Divalent (also called bivalent) scFvs can be generated by linking two scFvs. For example, a divalent scFv can be made by generating a single peptide containing two V_{H} and two V_{L} regions. Alternatively, two peptides, each containing a single V_{H} and a single V_{L} region can be dimerized (also called “diabodies”). Holliger et al., “Diabodies: small bivalent and bispecific antibody fragments,” PNAS, July 1993, 90:6444-6448. Bivalency allows antibodies to bind to multimeric antigens with high avidity, and bispecificity allows the cross-linking of two antigens.

As used herein, the term “monoclonal antibody” refers to an antibody obtained from a group of substantially homogeneous antibodies, that is, an antibody group wherein the antibodies constituting the group are homogeneous except for naturally occurring mutants that exist in a small amount. Monoclonal antibodies are highly specific and interact with a single antigenic site. Furthermore, each monoclonal antibody targets a single antigenic determinant (epitope) on an antigen, as compared to common polyclonal antibody preparations that typically contain various antibodies against diverse antigenic determinants. In addition to their specificity, monoclonal antibodies are advantageous in that they are typically produced from hybridoma cultures not contaminated with other immunoglobulins.

The adjective “monoclonal” indicates a characteristic of antibodies obtained from a substantially homogeneous group of antibodies, and does not specify antibodies produced by a particular method. For example, a monoclonal antibody to be used in the present invention can be produced by, for example, hybridoma methods (Kohler and Milstein, Nature 256:495, 1975) or recombination methods (U.S. Pat. No. 4,816,567). The monoclonal antibodies used in the present invention can be also isolated from a phage antibody library (Clackson et al., Nature 352:624-628, 1991; Marks et al., J. Mol. Biol. 222:581-597, 1991). The monoclonal antibodies of the present invention may comprise “chimeric” antibodies (immunoglobulins), wherein a part of a heavy (H) chain and/or light (L) chain is derived from a specific species or a specific antibody class or subclass, and the remaining portion of the chain is derived from another species, or another antibody class or subclass. Furthermore, mutant antibodies and antibody fragments thereof are also comprised in the present invention (U.S. Pat. No. 4,816,567; Morrison et al., Proc. Natl. Acad. Sci. USA 81:6851-6855, 1984).

As used herein, the term “mutant antibody” refers to an antibody comprising a variant amino acid sequence in which one or more amino acid residues have been altered. For example, the variable region of an antibody can be modified to improve its biological properties, such as antigen binding. Such modifications can be achieved by site-directed mutagenesis (see Kunkel, Proc. Natl. Acad. Sci. USA 82: 488 (1985)), PCR-based mutagenesis, cassette mutagenesis, and the like. Such mutants comprise an amino acid sequence which is at least 70% identical to the amino acid sequence of a heavy or light chain variable region of the antibody, more specifically at least 75%, even more specifically at least 80%, still more specifically at least 85%, yet more specifically at least 90%, and most specifically at least 95% identical. As used herein, the term “sequence identity” is defined as the percentage of residues identical to those in the antibody's original amino acid sequence, determined after the sequences are aligned and gaps are appropriately introduced to maximize the sequence identity as necessary.

Specifically, the identity of one nucleotide sequence or amino acid sequence to another can be determined using the algorithm BLAST, by Karlin and Altschul (Proc. Natl. Acad. Sci. USA, 90: 5873-5877, 1993). Programs such as BLASTN and BLASTX were developed based on this algorithm (Altschul et al., J. Mol. Biol. 215: 403-410, 1990). To analyze nucleotide sequences according to BLASTN based on BLAST, the parameters are set, for example, as score=100 and wordlength=12. On the other hand, parameters used for the analysis of amino acid sequences by BLASTX based on BLAST include, for example, score=50 and wordlength=3. Default parameters for each program are used when using the BLAST and Gapped BLAST programs. Specific techniques for such analyses are known in the art (see the website of the National Center for Biotechnology Information (NCBI), Basic Local Alignment Search Tool (BLAST); http://www.ncbi.nlm.nih.gov).

Polyclonal and monoclonal antibodies can be prepared by methods known to those skilled in the art.

In another embodiment, antibodies or antibody fragments can be isolated from an antibody phage library, produced by using the technique reported by McCafferty et al. (Nature 348:552-554 (1990)). Clackson et al. (Nature 352:624-628 (1991)) and Marks et al. (J. Mol. Biol. 222:581-597 (1991)) reported on the respective isolation of mouse and human antibodies from phage libraries. There are also reports that describe the production of high affinity (nM range) human antibodies based on chain shuffling (Marks et al., Bio/Technology 10:779-783 (1992)), and combinatorial infection and in vivo recombination, which are methods for constructing large-scale phage libraries (Waterhouse et al., Nucleic Acids Res. 21:2265-2266 (1993)). These technologies can also be used to isolate monoclonal antibodies, instead of using conventional hybridoma technology for monoclonal antibody production.

Antibodies to be used in the present invention can be purified by a method appropriately selected from known methods, such as the protein A-Sepharose method, hydroxyapatite chromatography, salting-out method with sulfate, ion exchange chromatography, and affinity chromatography, or by the combined use of the same.

The present invention may use recombinant antibodies, produced by gene engineering. The genes encoding the antibodies obtained by a method described above are isolated from the hybridomas. The genes are inserted into an appropriate vector, and then introduced into a host (see, e.g., Carl, A. K. Borrebaeck, James, W. Larrick, Therapeutic Monoclonal Antibodies, Published in the United Kingdom by Macmillan Publishers Ltd, 1990). The present invention provides the nucleic acids encoding the antibodies of the present invention, and vectors comprising these nucleic acids. Specifically, using a reverse transcriptase, cDNAs encoding the variable regions (V regions) of the antibodies are synthesized from the mRNAs of hybridomas. After obtaining the DNAs encoding the variable regions of antibodies of interest, they are ligated with DNAs encoding desired constant regions (C regions) of the antibodies, and the resulting DNA constructs are inserted into expression vectors. Alternatively, the DNAs encoding the variable regions of the antibodies may be inserted into expression vectors comprising the DNAs of the antibody C regions. These are inserted into expression vectors so that the genes are expressed under the regulation of an expression regulatory region, for example, an enhancer and promoter. Then, host cells are transformed with the expression vectors to express the antibodies. The present invention provides cells expressing antibodies of the present invention. The cells expressing antibodies of the present invention include cells and hybridomas transformed with a gene of such an antibody.

The antibodies of the present invention also include antibodies which comprise complementarity-determining regions (CDRs), or regions functionally equivalent to CDRs. The term “functionally equivalent” refers to comprising amino acid sequences similar to the amino acid sequences of CDRs of any of the monoclonal antibodies isolated in the Examples. The term “CDR” refers to a region in an antibody variable region (also called “V region”), and determines the specificity of antigen binding. The H chain and L chain each have three CDRs, designated from the N terminus as CDR1, CDR2, and CDR3. There are four regions flanking these CDRs: these regions are referred to as “framework,” and their amino acid sequences are highly conserved. The CDRs can be transplanted into other antibodies, and thus a recombinant antibody can be prepared by combining CDRs with the framework of a desired antibody. One or more amino acids of a CDR can be modified without losing the ability to bind to its antigen. For example, one or more amino acids in a CDR can be substituted, deleted, and/or added.

In certain embodiments, an amino acid residue is mutated into one that allows the properties of the amino acid side-chain to be conserved. Examples of the properties of amino acid side chains comprise: hydrophobic amino acids (A, I, L, M, F, P, W, Y, V), hydrophilic amino acids (R, D, N, C, E, Q, G, H, K, S, T), and amino acids comprising the following side chains: aliphatic side-chains (G, A, V, L, I, P); hydroxyl group-containing side-chains (S, T, Y); sulfur atom-containing side-chains (C, M); carboxylic acid- and amide-containing side-chains (D, N, E, Q); base-containing side-chains (R, K, H); and aromatic-containing side-chains (H, F, Y, W). The letters within parenthesis indicate the one-letter amino acid codes. Amino acid substitutions within each group are called conservative substitutions. It is well known that a polypeptide comprising a modified amino acid sequence in which one or more amino acid residues is deleted, added, and/or substituted can retain the original biological activity (Mark D. F. et al., Proc. Natl. Acad. Sci. U.S.A. 81:5662-5666 (1984); Zoller M. J. and Smith M., Nucleic Acids Res. 10: 6487-6500 (1982); Wang A. et al., Science 224: 1431-1433; Dalbadie-McFarland G. et al., Proc. Natl. Acad. Sci. U.S.A. 79: 6409-6413 (1982)). The number of mutated amino acids is not limited, but in general, the number falls within 40% of amino acids of each CDR, and specifically within 35%, and still more specifically within 30% (e.g., within 25%). The identity of amino acid sequences can be determined as described herein.

In the present invention, recombinant antibodies artificially modified to reduce heterologous antigenicity against humans can be used. Examples include chimeric antibodies and humanized antibodies. These modified antibodies can be produced using known methods. A chimeric antibody includes an antibody comprising variable and constant regions of species that are different to each other, for example, an antibody comprising the antibody heavy chain and light chain variable regions of a nonhuman mammal such as a mouse, and the antibody heavy chain and light chain constant regions of a human. Such an antibody can be obtained by (1) ligating a DNA encoding a variable region of a mouse antibody to a DNA encoding a constant region of a human antibody; (2) incorporating this into an expression vector; and (3) introducing the vector into a host for production of the antibody.

A humanized antibody, which is also called a reshaped human antibody, may be obtained by substituting an H or L chain complementarity determining region (CDR) of an antibody of a nonhuman mammal such as a mouse, with the CDR of a human antibody. Conventional genetic recombination techniques for the preparation of such antibodies are known (see, for example, Jones et al., Nature 321: 522-525 (1986); Reichmann et al., Nature 332: 323-329 (1988); Presta Curr. Op. Struct. Biol. 2: 593-596 (1992)). Specifically, a DNA sequence designed to ligate a CDR of a mouse antibody with the framework regions (FRs) of a human antibody is synthesized by PCR, using several oligonucleotides constructed to comprise overlapping portions at their ends. A humanized antibody can be obtained by (1) ligating the resulting DNA to a DNA that encodes a human antibody constant region; (2) incorporating this into an expression vector; and (3) transfecting the vector into a host to produce the antibody (see, European Patent Application No. EP 239,400, and International Patent Application No. WO 96/02576). Human antibody FRs that are ligated via the CDR are selected where the CDR forms a favorable antigen-binding site. The humanized antibody may comprise additional amino acid residue(s) that are not included in the CDRs introduced into the recipient antibody, nor in the framework sequences. Such amino acid residues are usually introduced to more accurately optimize the antibody's ability to recognize and bind to an antigen. For example, as necessary, amino acids in the framework region of an antibody variable region may be substituted such that the CDR of a reshaped human antibody forms an appropriate antigen-binding site (Sato, K. et al., Cancer Res. (1993) 53, 851-856).

As described herein, an antibody of the invention or a fragment thereof may comprise a synthetic CDR-H3. Additionally, an antibody of the invention may also be a recombinant antibody (e.g., a humanized or chimeric antibody) or a fragment thereof. Accordingly, such an antibody of the invention or fragment thereof would not be a product of nature. Additionally, an antibody of the invention or a fragment thereof may comprise markedly different characteristics (e.g., structural, functional and/or other properties) as compared to naturally occurring antibody.

The isotypes of the antibodies of the present invention are not limited. The isotypes include, for example, IgG (IgG1, IgG2, IgG3, and IgG4), IgM, IgA (IgA1 and IgA2), IgD, and IgE. The antibodies of the present invention may also be antibody fragments comprising a portion responsible for antigen binding, or a modified fragment thereof. The term “antibody fragment” refers to a portion of a full-length antibody, and generally to a fragment comprising an antigen-binding domain or a variable region. Such antibody fragments include, for example, Fab, F(ab′)₂, Fv, single-chain Fv (scFv) which comprises a heavy chain Fv and a light chain Fv coupled together with an appropriate linker, diabody (diabodies), linear antibodies, and multispecific antibodies prepared from antibody fragments. Previously, antibody fragments were produced by digesting natural antibodies with a protease; currently, methods for expressing them as recombinant antibodies using genetic engineering techniques are also known (see Morimoto et al., Journal of Biochemical and Biophysical Methods 24:107-117 (1992); Brennan et al., Science 229:81 (1985); Co, M. S. et al., J. Immunol., 1994, 152, 2968-2976; Better, M. & Horwitz, A. H., Methods in Enzymology, 1989, 178, 476-496, Academic Press, Inc.; Plueckthun, A. & Skerra, A., Methods in Enzymology, 1989, 178, 476-496, Academic Press, Inc.; Lamoyi, E., Methods in Enzymology, 1989, 121, 663-669; Bird, R. E. et al., TIBTECH, 1991, 9, 132-137).

An “Fv” fragment is the smallest antibody fragment, and contains a complete antigen recognition site and a binding site. This region is a dimer (V_{H}-V_{L} dimer) wherein the variable regions of each of the heavy chain and light chain are strongly connected by a noncovalent bond. The three CDRs of each of the variable regions interact with each other to form an antigen-binding site on the surface of the V_{H}-V_{L} dimer. In other words, a total of six CDRs from the heavy and light chains function together as an antibody's antigen-binding site. However, a variable region (or a half Fv, which contains only three antigen-specific CDRS) alone is also known to be able to recognize and bind to an antigen, although its affinity is lower than the affinity of the entire binding site. Thus, a specific antibody fragment of the present invention is an Fv fragment, but is not limited thereto. Such an antibody fragment may be a polypeptide which comprises an antibody fragment of heavy or light chain CDRs which are conserved, and which can recognize and bind its antigen.

A Fab fragment (also referred to as F(ab)) also contains a light chain constant region and heavy chain constant region (CH1). For example, papain digestion of an antibody produces the two kinds of fragments: an antigen-binding fragment, called a Fab fragment, containing the variable regions of a heavy chain and light chain, which serve as a single antigen-binding domain; and the remaining portion, which is called an “Fc” because it is readily crystallized. A Fab′ fragment is different from a Fab fragment in that a Fab′ fragment also has several residues derived from the carboxyl terminus of a heavy chain CH1 region, which contains one or more cysteine residues from the hinge region of an antibody. A Fab′ fragment is, however, structurally equivalent to Fab in that both are antigen-binding fragments which comprise the variable regions of a heavy chain and light chain, which serve as a single antigen-binding domain. Herein, an antigen-binding fragment comprising the variable regions of a heavy chain and light chain which serve as a single antigen-binding domain, and which is equivalent to that obtained by papain digestion, is referred to as a “Fab-like antibody,” even when it is not identical to an antibody fragment produced by protease digestion. Fab′-SH is Fab′ with one or more cysteine residues having free thiol groups in its constant region. A F(ab′) fragment is produced by cleaving the disulfide bond between the cysteine residues in the hinge region of F(ab′)₂. Other chemically crosslinked antibody fragments are also known to those skilled in the art. Pepsin digestion of an antibody yields two fragments; one is a F(ab′)₂ fragment which comprises two antigen-binding domains and can cross-react with antigens, and the other is the remaining fragment (referred to as pFc′). Herein, an antibody fragment equivalent to that obtained by pepsin digestion is referred to as a “F(ab′)₂-like antibody” when it comprises two antigen-binding domains and can cross-react with antigens. Such antibody fragments can also be produced, for example, by genetic engineering. Such antibody fragments can also be isolated, for example, from the antibody phage library described above. Alternatively, F(ab′)₂-SH fragments can be recovered directly from hosts, such as E. coli, and then allowed to form F(ab′)₂ fragments by chemical crosslinking (Carter et al., Bio/Technology 10:163-167 (1992)). In an alternative method, F(ab′)₂ fragments can be isolated directly from a culture of recombinant hosts.

The term “diabody (db)” refers to a bivalent antibody fragment constructed by gene fusion (for example, P. Holliger et al., Proc. Natl. Acad. Sci. USA 90: 6444-6448 (1993), EP 404,097, WO 93/11161). In general, a diabody is a dimer of two polypeptide chains. In the each of the polypeptide chains, a light chain variable region (V_{L}) and a heavy chain variable region (V_{H}) in an identical chain are connected via a short linker, for example, a linker of about five residues, so that they cannot bind together. Because the linker between the two is too short, the V_{L} and V_{H} in the same polypeptide chain cannot form a single chain V region fragment, but instead form a dimer. Thus, a diabody has two antigen-binding domains. When the V_{L} and V_{H} regions against the two types of antigens (a and b) are combined to form V_{La}-V_{Hb} and V_{Lb}-V_{Ha} via a linker of about five residues, and then co-expressed, they are secreted as bispecific Dbs. The antibodies of the present invention may be such Dbs.

A single-chain antibody (also referred to as “scFv”) can be prepared by linking a heavy chain V region and a light chain V region of an antibody (for a review of scFv see Pluckthun “The Pharmacology of Monoclonal Antibodies” Vol. 113, eds. Rosenburg and Moore, Springer Verlag, N.Y., pp. 269-315 (1994)). Methods for preparing single-chain antibodies are known in the art (see, for example, U.S. Pat. Nos. 4,946,778; 5,260,203; 5,091,513; and 5,455,030). In such scFvs, the heavy chain V region and the light chain V region are linked together via a linker, such as a polypeptide linker (Huston, J. S. et al., Proc. Natl. Acad. Sci. U.S.A, 1988, 85, 5879-5883). The heavy chain V region and the light chain V region in a scFv may be derived from the same antibody, or from different antibodies. The peptide linker used to ligate the V regions may be any single-chain peptide consisting of 12 to 19 residues. A DNA encoding a scFv can be amplified by PCR using, as a template, either the entire DNA, or a partial DNA encoding a desired amino acid sequence, selected from a DNA encoding the heavy chain or the V region of the heavy chain of the above antibody, and a DNA encoding the light chain or the V region of the light chain of the above antibody; and using a primer pair that defines the two ends. Further amplification can be subsequently conducted using a combination of the DNA encoding the peptide linker portion, and the primer pair that defines both ends of the DNA to be ligated to the heavy and light chain respectively. After constructing DNAs encoding scFvs, conventional methods can be used to obtain expression vectors comprising these DNAs, and hosts transformed by these expression vectors. Furthermore, scFvs can be obtained according to conventional methods using the resulting hosts. These antibody fragments can be produced in hosts by obtaining genes that encode the antibody fragments and expressing these as outlined above. Antibodies bound to various types of molecules, such as polyethylene glycols (PEGs), may be used as modified antibodies. Methods for modifying antibodies are already established in the art. The term “antibody” in the present invention also encompasses the above-described antibodies.

The antibodies obtained can be purified to homogeneity. The antibodies can be isolated and purified by a method routinely used to isolate and purify proteins. The antibodies can be isolated and purified by the combined use of one or more methods appropriately selected from column chromatography, filtration, ultrafiltration, salting out, dialysis, preparative polyacrylamide gel electrophoresis, and isoelectro-focusing, for example (Strategies for Protein Purification and Characterization: A Laboratory Course Manual, Daniel R. Marshak et al. eds., Cold Spring Harbor Laboratory Press (1996); Antibodies: A Laboratory Manual. Ed Harlow and David Lane, Cold Spring Harbor Laboratory, 1988). Such methods are not limited to those listed above. Chromatographic methods include affinity chromatography, ion exchange chromatography, hydrophobic chromatography, gel filtration, reverse-phase chromatography, and adsorption chromatography. These chromatographic methods can be practiced using liquid phase chromatography, such as HPLC and FPLC. Columns to be used in affinity chromatography include protein A columns and protein G columns. For example, protein A columns include Hyper D, POROS, and Sepharose F. F. (Pharmacia). Antibodies can also be purified by utilizing antigen binding, using carriers on which antigens have been immobilized.

The antibodies of the present invention can be formulated according to standard methods (see, for example, Remington's Pharmaceutical Science, latest edition, Mark Publishing Company, Easton, U.S.A), and may comprise pharmaceutically acceptable carriers and/or additives. The present invention relates to compositions (including reagents and pharmaceuticals) comprising the antibodies of the invention, and pharmaceutically acceptable carriers and/or additives. Exemplary carriers include surfactants (for example, PEG and Tween), excipients, antioxidants (for example, ascorbic acid), coloring agents, flavoring agents, preservatives, stabilizers, buffering agents (for example, phosphoric acid, citric acid, and other organic acids), chelating agents (for example, EDTA), suspending agents, isotonizing agents, binders, disintegrators, lubricants, fluidity promoters, and corrigents. However, the carriers that may be employed in the present invention are not limited to this list. In fact, other commonly used carriers can be appropriately employed: light anhydrous silicic acid, lactose, crystalline cellulose, mannitol, starch, carmelose calcium, carmelose sodium, hydroxypropylcellulose, hydroxypropylmethyl cellulose, polyvinylacetaldiethylaminoacetate, polyvinylpyrrolidone, gelatin, medium chain fatty acid triglyceride, polyoxyethylene hydrogenated castor oil 60, sucrose, carboxymethylcellulose, corn starch, inorganic salt, and so on. The composition may also comprise other low-molecular-weight polypeptides, proteins such as serum albumin, gelatin, and immunoglobulin, and amino acids such as glycine, glutamine, asparagine, arginine, and lysine. When the composition is prepared as an aqueous solution for injection, it can comprise an isotonic solution comprising, for example, physiological saline, dextrose, and other adjuvants, including, for example, D-sorbitol, D-mannose, D-mannitol, and sodium chloride, which can also contain an appropriate solubilizing agent, for example, alcohol (for example, ethanol), polyalcohol (for example, propylene glycol and PEG), and non-ionic detergent (polysorbate 80 and HCO-50).

If necessary, antibodies of the present invention may be encapsulated in microcapsules (microcapsules made of hydroxycellulose, gelatin, polymethylmethacrylate, and the like), and made into components of colloidal drug delivery systems (liposomes, albumin microspheres, microemulsions, nano-particles, and nano-capsules) (for example, see “Remington's Pharmaceutical Science 16th edition”, Oslo Ed. (1980)). Moreover, methods for making sustained-release drugs are known, and these can be applied for the antibodies of the present invention (Langer et al., J. Biomed. Mater. Res. 15: 167-277 (1981); Langer, Chem. Tech. 12: 98-105 (1982); U.S. Pat. No. 3,773,919; EP Patent Application No. 58,481; Sidman et al., Biopolymers 22: 547-556 (1983); EP: 133,988).

### Nucleic Acids, Expression Cassettes, Vectors and Cells

Certain embodiments of the invention provide an isolated nucleic acid encoding an antibody or fragment thereof as described herein. In certain embodiments, the nucleic acid further comprises a promoter.

Certain embodiments of the invention provide an expression cassette comprising a nucleic acid as described herein and a promoter.

Certain embodiments of the invention provide a vector (e.g., a phagemid) comprising a nucleic acid or an expression cassette as described herein.

Certain embodiments of the invention provide a cell comprising a nucleic acid, expression cassette or vector as described herein.

Certain embodiments of the invention provide a phage particle comprising a vector as described herein.

The term “nucleic acid” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucl. Acids Res., 19:508 (1991); Ohtsuka et al., JBC, 260:2605 (1985); Rossolini et al., Mol. Cell. Probes, 8:91 (1994). A “nucleic acid fragment” is a fraction of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA that can be single- or double-stranded, optionally containing synthetic, non-natural or altered nucleotide bases capable of incorporation into DNA or RNA polymers. The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene.

By “portion” or “fragment,” as it relates to a nucleic acid molecule, sequence or segment of the invention, when it is linked to other sequences for expression, is meant a sequence having at least 80 nucleotides, more specifically at least 150 nucleotides, and still more specifically at least 400 nucleotides. If not employed for expressing, a “portion” or “fragment” means at least 9, specifically 12, more specifically 15, even more specifically at least 20, consecutive nucleotides, e.g., probes and primers (oligonucleotides), corresponding to the nucleotide sequence of the nucleic acid molecules of the invention.

The terms “protein,” “peptide” and “polypeptide” are used interchangeably herein.

The invention encompasses isolated or substantially purified nucleic acid or protein compositions. In the context of the present invention, an “isolated” or “purified” DNA molecule or an “isolated” or “purified” polypeptide is a DNA molecule or polypeptide that exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, culture medium may represent less than about 30%, 20%, 10%, or 5% (by dry weight) of chemical precursors or non-protein-of-interest chemicals. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein.

“Naturally occurring” is used to describe an object that can be found in nature as distinct from being artificially produced. For example, a protein or nucleotide sequence present in an organism (including a virus), which can be isolated from a source in nature and which has not been intentionally modified by man in the laboratory, is naturally occurring.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have at least 40, 50, 60, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations” which are one species of “conservatively modified variations.” Every nucleic acid sequence described herein which encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a polypeptide is implicit in each described sequence.

“Recombinant DNA molecule” is a combination of DNA sequences that are joined together using recombinant DNA technology and procedures used to join together DNA sequences as described, for example, in Sambrook and Russell, Molecular Cloning: A Laboratory Manual, Cold Spring Harbor, N.Y.: Cold Spring Harbor Laboratory Press (3^{rd} edition, 2001).

The terms “heterologous DNA sequence,” “exogenous DNA segment” or “heterologous nucleic acid,” each refer to a sequence that originates from a source foreign to the particular host cell or, if from the same source, is modified from its original form. Thus, a heterologous gene in a host cell includes a gene that is endogenous to the particular host cell but has been modified. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” DNA sequence is a DNA sequence that is naturally associated with a host cell into which it is introduced.

“Wild-type” refers to the normal gene, or organism found in nature without any known mutation.

“Genome” refers to the complete genetic material of an organism.

A “vector” is defined to include, inter alia, any plasmid, cosmid, phage or binary vector in double or single stranded linear or circular form which may or may not be self transmissible or mobilizable, and which can transform prokaryotic or eukaryotic host either by integration into the cellular genome or exist extrachromosomally (e.g., autonomous replicating plasmid with an origin of replication).

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. The expression of the nucleotide sequence in the expression cassette may be under the control of a constitutive promoter or of an inducible promoter that initiates transcription only when the host cell is exposed to some particular external stimulus. In the case of a multicellular organism, the promoter can also be specific to a particular tissue or organ or stage of development.

Such expression cassettes will comprise the transcriptional initiation region of the invention linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted above, the term “suitable regulatory sequences” is not limited to promoters. However, some suitable regulatory sequences useful in the present invention will include, but are not limited to constitutive promoters, tissue-specific promoters, development-specific promoters, inducible promoters and viral promoters.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency (Turner et al., Mol. Biotech., 3:225 (1995).

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

The term “mature” protein refers to a post-translationally processed polypeptide without its signal peptide. “Precursor” protein refers to the primary product of translation of an mRNA. “Signal peptide” refers to the amino terminal extension of a polypeptide, which is translated in conjunction with the polypeptide forming a precursor peptide and which is required for its entrance into the secretory pathway. The term “signal sequence” refers to a nucleotide sequence that encodes the signal peptide.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

The “initiation site” is the position surrounding the first nucleotide that is part of the transcribed sequence, which is also defined as position +1. With respect to this site all other sequences of the gene and its controlling regions are numbered. Downstream sequences (i.e. further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Constitutive expression” refers to expression using a constitutive or regulated promoter. “Conditional” and “regulated expression” refer to expression controlled by a regulated promoter.

“Operably-linked” may refer to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation in a cell of an endogenous gene, transgene, as well as the transcription and stable accumulation of sense (mRNA) or functional RNA. In the case of antisense constructs, expression may refer to the transcription of the antisense DNA only. Expression may also refer to the production of protein.

“Transcription stop fragment” refers to nucleotide sequences that contain one or more regulatory signals, such as polyadenylation signal sequences, capable of terminating transcription. Examples of transcription stop fragments are known to the art.

“Translation stop fragment” refers to nucleotide sequences that contain one or more regulatory signals, such as one or more termination codons in all three frames, capable of terminating translation. Insertion of a translation stop fragment adjacent to or near the initiation codon at the 5′ end of the coding sequence will result in no translation or improper translation. Excision of the translation stop fragment by site-specific recombination will leave a site-specific sequence in the coding sequence that does not interfere with proper translation using the initiation codon.

The terms “cis-acting sequence” and “cis-acting element” refer to DNA or RNA sequences whose functions require them to be on the same molecule.

The terms “trans-acting sequence” and “trans-acting element” refer to DNA or RNA sequences whose function does not require them to be on the same molecule.

The following terms are used to describe the sequence relationships between two or more sequences (e.g., nucleic acids, polynucleotides or polypeptides): (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full length cDNA, gene sequence or peptide sequence, or the complete cDNA, gene sequence or peptide sequence.

(b) As used herein, “comparison window” makes reference to a contiguous and specified segment of a sequence, wherein the sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the sequence a gap penalty is typically introduced and is subtracted from the number of matches.

Methods of alignment of sequences for comparison are well known in the art. Thus, the determination of percent identity between any two sequences can be accomplished using a mathematical algorithm. Non-limiting examples of such mathematical algorithms are the algorithm of Myers and Miller, CABIOS, 4:11 (1988); the local homology algorithm of Smith et al., Adv. Appl. Math., 2:482 (1981); the homology alignment algorithm of Needleman and Wunsch, JMB, 48:443 (1970); the search-for-similarity-method of Pearson and Lipman, Proc. Natl. Acad. Sci. USA, 85:2444 (1988); the algorithm of Karlin and Altschul, Proc. Natl. Acad. Sci. USA, 87:2264 (1990), modified as in Karlin and Altschul, Proc. Natl. Acad. Sci. USA, 90:5873 (1993).

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al., Gene, 73:237 (1988); Higgins et al., CABIOS, 5:151 (1989); Corpet et al., Nucl. Acids Res., 16:10881 (1988); Huang et al., CABIOS, 8:155 (1992); and Pearson et al., Meth. Mol. Biol., 24:307 (1994). The ALIGN program is based on the algorithm of Myers and Miller, supra. The BLAST programs of Altschul et al., JMB, 215:403 (1990); Nucl. Acids Res., 25:3389 (1990), are based on the algorithm of Karlin and Altschul supra.

Software for performing BLAST analyses is publicly available through the National Center for Biotechnology Information (available on the world wide web at ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more specifically less than about 0.01, and most specifically less than about 0.001.

To obtain gapped alignments for comparison purposes, Gapped BLAST (in BLAST 2.0) can be utilized as described in Altschul et al., Nucleic Acids Res. 25:3389 (1997). Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See the world wide web at ncbi.nlm.nih.gov. Alignment may also be performed manually by visual inspection.

For purposes of the present invention, comparison of sequences for determination of percent sequence identity to another sequence may be made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of sequences means that a polynucleotide comprises a sequence that has at least 70%, 71%, 72%, 73%, 74%, 75%, 76%, 77%, 78%, or 79%, at least 80%, 81%, 82%, 83%, 84%, 85%, 86%, 87%, 88%, or 89%, at least 90%, 91%, 92%, 93%, or 94%, and at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, at least 95%.

Another indication that nucleotide sequences are substantially identical is if two molecules hybridize to each other under stringent conditions (see below). Generally, stringent conditions are selected to be about 5° C. lower than the thermal melting point (Tm) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, or 79%, 80%, 81%, 82%, 83%, 84%, 85%, 86%, 87%, 88%, or 89%, at least 90%, 91%, 92%, 93%, or 94%, or 95%, 96%, 97%, 98% or 99%, sequence identity to the reference sequence over a specified comparison window. Optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The thermal melting point (Tm) is the temperature (under defined ionic strength and pH) at which 50% of the target sequence hybridizes to a perfectly matched probe. Specificity is typically the function of post-hybridization washes, the critical factors being the ionic strength and temperature of the final wash solution.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or from human manipulation. Methods for such manipulations are generally known in the art.

Thus, the polypeptides of the invention may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. See, for example, Kunkel, Proc. Natl. Acad. Sci. USA, 82:488 (1985); Kunkel et al., Meth. Enzymol., 154:367 (1987); U.S. Pat. No. 4,873,192; Walker and Gaastra, Techniques in Mol. Biol. (MacMillan Publishing Co. (1983), and the references cited therein. Guidance as to appropriate amino acid substitutions that do not affect biological activity of the protein of interest may be found in the model of Dayhoff et al., Atlas of Protein Sequence and Structure (Natl. Biomed. Res. Found. 1978). Conservative substitutions, such as exchanging one amino acid with another having similar properties, are preferred.

Thus, the genes and nucleotide sequences of the invention include both the naturally occurring sequences as well as mutant forms. Likewise, the polypeptides of the invention encompass naturally occurring proteins as well as variations and modified forms thereof. Such variants will continue to possess the desired activity. The deletions, insertions, and substitutions of the polypeptide sequence encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by routine screening assays.

Individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q). In addition, individual substitutions, deletions or additions which alter, add or delete a single amino acid or a small percentage of amino acids in an encoded sequence are also “conservatively modified variations.”

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism into which a heterologous nucleic acid molecule has been introduced. The nucleic acid molecule can be stably integrated into the genome generally known in the art and are disclosed in Sambrook and Russell, supra. See also Innis et al., PCR Protocols, Academic Press (1995); and Gelfand, PCR Strategies, Academic Press (1995); and Innis and Gelfand, PCR Methods Manual, Academic Press (1999). Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

### Methods of Use

Certain embodiments provide a method of detecting the presence of MMP-9 in a cell, the method comprising contacting the cell with an isolated anti-MMP-9 antibody, or fragment thereof, as described herein and detecting whether a complex is formed between the anti-MMP-9 antibody and MMP-9.

In certain embodiments, the cell is contacted in vitro. In certain embodiments, the cell is contacted in vivo.

Certain embodiments provide a method of inhibiting the activity of MMP-9 (e.g., protease cleavage activity), comprising contacting MMP-9 with an isolated anti-MMP-9 antibody, or fragment thereof, as described herein. In certain embodiments, MPP-9 collagenolysis is inhibited. In certain embodiments, the MMP-9 protein is contacted in vitro. In certain embodiments, the MMP-9 protein is contacted in vivo. Methods for measuring the activity of MMP-9 are known in the art. For example, in certain embodiments, an assay described herein may be used. In certain embodiments, an antibody of the invention or a fragment thereof inhibits the enzymatic activity of MMP-9 or collagenolysis by at least about 5%, at least about 10%, at least about 15%, at least about 20%, at least about 25%, at least about 30%, at least about 40%, at least about 50%, at least about 60%, at least about 70%, at least about 80%, at least about 90%, at least about 95%, at least about 99% or at least about 100% as compared to a control.

Certain embodiments also provide a method for treating pain in a mammal, comprising administering an effective amount of an isolated anti-MMP-9 antibody, or fragment thereof, as described herein to the mammal.

In certain embodiments, the method further comprises administering at least one additional therapeutic agent to the mammal. In certain embodiments, the at least one additional therapeutic agent is useful for treating pain. In certain embodiments, the at least one additional therapeutic agent is a steroid, a non-steroid anti-inflammatory drug (NSAIDs), a nerve blocker, an anti-depressant, gabapentin, Lyrica, a local anesthetic (e.g., lidocaine) or an opioid.

Certain embodiments provide an isolated anti-MMP-9 antibody, or fragment thereof, as described herein for the prophylactic or therapeutic treatment of pain.

Certain embodiments provide the use of an isolated anti-MMP-9 antibody, or fragment thereof, as described herein to prepare a medicament for the treatment of pain in a mammal.

In certain embodiments, the pain is chronic pain. In certain embodiments, the pain is neuropathic pain (e.g., associated with diabetes; associated with a viral infection, such as Shingles (Herpes Zoster) or an HIV infection; or associated with a surgery, such as a thoracotomy or amputation). In certain embodiments, the pain is associated with or results from chemotherapy, nerve injury, trigeminal neuralgia, spinal cord injury, stroke, brain trauma, arthritic pain (e.g., osteoarthritis or rheumatoid arthritis), headache or migraine, cancer or surgery (e.g., postoperative pain).

Certain embodiments also provide a method for treating a stroke in a mammal, comprising administering an effective amount of an isolated anti-MMP-9 antibody, or fragment thereof, as described herein to the mammal.

In certain embodiments, the method further comprises administering at least one additional therapeutic agent to the mammal. In certain embodiments, the at least one additional therapeutic agent is useful for treating a stroke.

Certain embodiments provide an isolated anti-MMP-9 antibody, or fragment thereof, as described herein for the prophylactic or therapeutic treatment of a stroke.

Certain embodiments provide the use of an isolated anti-MMP-9 antibody, or fragment thereof, as described herein to prepare a medicament for the treatment of a stroke in a mammal.

Certain embodiments provide an isolated anti-MMP-9 antibody, or fragment thereof, as described herein for use in medical therapy.

### Administration

For in vivo use, an antibody of the invention, or fragment thereof, is generally incorporated into a pharmaceutical composition prior to administration. Within such compositions, one or more antibodies of the invention may be present as active ingredient(s) (i.e., are present at levels sufficient to provide a statistically significant effect on the symptoms of a relevant disease (e.g., pain), as measured using a representative assay). A pharmaceutical composition comprises one or more such antibodies in combination with any pharmaceutically acceptable carrier(s) known to those skilled in the art to be suitable for the particular mode of administration. In addition, other pharmaceutically active ingredients (including other therapeutic agents) may, but need not, be present within the composition.

The term “therapeutically effective amount,” in reference to treating a disease state/condition, refers to an amount of an antibody or fragment thereof either alone or as contained in a pharmaceutical composition that is capable of having any detectable, positive effect on any symptom, aspect, or characteristics of a disease state/condition when administered as a single dose or in multiple doses. Such effect need not be absolute to be beneficial.

The terms “treat” and “treatment” refer to both therapeutic treatment and prophylactic or preventative measures, wherein the object is to prevent or decrease an undesired physiological change or disorder, such as pain. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

In certain embodiments, the present antibodies (i.e., antibody of the present invention or a fragment thereof) may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the antibody may be combined with one or more excipients and used in the form of ingestible tablets, buccal tablets, troches, capsules, elixirs, suspensions, syrups, wafers, and the like. Such compositions and preparations should contain at least 0.1% of an antibody of the present invention. The percentage of the compositions and preparations may, of course, be varied and may conveniently be between about 2 to about 60% of the weight of a given unit dosage form. The amount of antibody in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the antibody, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the antibody may be incorporated into sustained-release preparations and devices.

The antibody may also be administered intravenously or intraperitoneally by infusion or injection. Additionally, the antibody may be administered by local injection, such as by intrathecal injection, epidural injection or peri-neural injection using a scope. Solutions of the antibody may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the antibody that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be useful to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the antibody in the required amount in the appropriate solvent with various of the other ingredients enumerated above, as required, followed by filter sterilization. In the case of sterile powders for the preparation of sterile injectable solutions, the methods of preparation are vacuum drying and the freeze drying techniques, which yield a powder of the antibody plus any additional desired ingredient present in the previously sterile-filtered solutions.

For topical administration, the present antibodies may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present antibodies can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions that can be used to deliver the antibodies of the present invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the antibodies of the present invention can be determined by comparing their in vitro activity, and in vivo activity in animal models. Methods for the extrapolation of effective dosages in mice, and other animals, to humans are known to the art; for example, see U.S. Pat. No. 4,938,949.

The amount of an antibody of the present invention required for use in treatment will vary with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

Antibodies of the invention can also be administered in combination with other therapeutic agents and/or treatments, such as other agents or treatments that are useful for the treatment of pain or stroke. Non-limiting examples of such agents include steroids, non-steroid anti-inflammatory drugs (NSAIDs), nerve blockers, anti-depressants, gabapentin, Lyrica, local anesthetics (e.g., lidocaine) and opioids. Additionally, one or more antibodies of the invention, or fragments thereof, may be administered (e.g., a combination of antibodies, or fragments thereof, may be administered). Accordingly, one embodiment the invention also provides a composition comprising an antibody of the invention, or a fragment thereof, at least one other therapeutic agent, and a pharmaceutically acceptable diluent or carrier. The invention also provides a kit comprising an antibody of the invention, or a fragment thereof, at least one other therapeutic agent, packaging material, and instructions for administering an antibody of the invention, or a fragment thereof, and the other therapeutic agent or agents to an animal to treat pain or stroke.

As used herein, the term “therapeutic agent” refers to any agent or material that has a beneficial effect on the mammalian recipient.

### Screening Methods

Current antibody discovery methods typically rely on the capability of antibody to bind to a target protein rather than the capability of the antibody to modulate the function of the target protein. Therefore, the identification of antibodies having inhibitory capabilities can be challenging using traditional techniques. As described herein, an alternative method for identifying and selecting antibodies having inhibitory functions has been developed.

Accordingly, certain embodiments of the invention provide a method of isolating an antibody or a fragment thereof from an antibody library, wherein the antibody or fragment thereof is capable of inhibiting a target protease, the method comprising: periplasmically co-expressing in a bacterial cell: 1) an antibody, or fragment thereof, from the library; 2) the target protease or an enzymatic domain thereof (e.g., extracellular or catalytic domain); and 3) a modified β-lactamase that comprises a peptide sequence that is capable of being cleaved by the target protease; wherein the bacterial cell is cultured in the presence of a β-lactam antibiotic. In such a method, an antibody or fragment thereof, that is capable of inhibiting the target protease will block the enzymatic activity of the target protease and prevent the cleavage of the 3-lactamase, resulting in cell growth in the presence of a β-lactam antibiotic. Alternatively, if an antibody or fragment thereof is not capable of inhibiting the target protease, the β-lactamase will be cleaved, leading to cell death in the presence of a β-lactam antibiotic.

In certain embodiments, such a method is repeated 2 or more times.

In certain embodiments, the method further comprises transfecting one or more plasmids comprising a nucleic acid encoding the antibody, or fragment thereof, a nucleic acid encoding the target protease, or domain thereof, and a nucleic acid encoding the modified β-lactamase into the bacterial cell. In certain embodiments, nucleic acids encoding the antibody, the target protease and the modified β-lactamase are present in a single plasmid. In certain embodiments, a bacterial cell is transfected with two plasmids, wherein the first plasmid comprises two different nucleic acids selected from the group consisting of a nucleic acid encoding the antibody, a nucleic acid encoding the target protease and a nucleic acid encoding the modified 3-lactamase, and wherein the second plasmid comprises a nucleic acid not present in the first plasmid selected from the group consisting of a nucleic acid encoding the antibody, a nucleic acid encoding the target protease and a nucleic acid encoding the modified β-lactamase. In certain embodiments, a bacterial cell is transfected with two plasmids, wherein the first plasmid comprises a nucleic acid encoding the target protease and a nucleic acid encoding the modified β-lactamase, and wherein the second plasmid comprises a nucleic acid encoding the antibody. In certain embodiments, a bacterial cell is transfected with a plasmid comprising a nucleic acid encoding the antibody, a plasmid comprising a nucleic acid encoding the target protease and a plasmid comprising a nucleic acid encoding the modified β-lactamase.

In certain embodiments, the bacterial cell is an Escherichia coli cell.

In certain embodiments, the bacterial cell is cultured for a time sufficient for cell growth or cell death to occur.

In certain embodiments, the modified β-lactamase is a modified TEM-1 (see, e.g., SEQ ID NOS 30 and 42). In certain embodiments, the cleavable peptide sequence is inserted between Gly196 and Glu197 of TEM-1 (see, e.g., SEQ ID NO:29). In certain embodiments, the modified β-lactamase is described herein. In certain embodiments, the cleavable peptide sequence is a sequence described herein (e.g., any one of SEQ ID NOs:31-32). In certain embodiments, a linker group is operably linked to the N′ and/or C′ terminus of the cleavable peptide sequence. In certain embodiments, the linker group is glycine/serine rich (e.g., SRGSGXSGGPW SEQ ID NOS 11 and 41, wherein “X” is the cleavable peptide sequence; see, SEQ ID NOS 30 and 42).

In certain embodiments, the bacterial cell is cultured in the presence of ampicillin.

In certain embodiments, the target protease is a matrix metalloproteinase (MMP). In certain embodiments, the MMP is a human MMP. In certain embodiments, the MMP is a MMP-1, MMP-2, MMP-3, MMP-7, MMP-8, MMP-9, MMP-10, MMP-11, MMP-12, MMP-13, MMP-14, MMP-15, MMP-16, MMP-17, MMP-19, MMP-20, MMP-21, MMP-23A, MMP-23B, MMP-24, MMP-25, MMP-26, MMP-27 or MMP-28. In certain embodiments, the MMP is MMP-2. In certain embodiments, the MMP is MMP-9. In certain embodiments, the MMP is MMP-14. In certain embodiments, the protease is BACE-1. In certain embodiments, the protease is Alp2. In certain embodiments, the protease is cathepsin B. In certain embodiments, the protease is capsase-6.

Certain embodiments of the invention provide an antibody, or fragment thereof, isolated by a method described herein.

Certain embodiments also provide a target protease inhibition polypeptide sensor comprising a β-lactamase TEM-1 amino acid sequence and a peptide sequence that is capable of being cleaved by a target protease, wherein the intact target protease inhibition polypeptide sensor is capable of hydrolyzing a β-lactam antibiotic. In certain embodiments, the cleavable peptide sequence is inserted between Gly196 and Glu197 of TEM-1 (see, e.g., SEQ ID NOs:29-30 and 42). In certain embodiments, the cleavable peptide sequence is a sequence described herein (e.g., any one of SEQ ID NOs:31-32). In certain embodiments, a linker group is operably linked to the N′ and/or C′ terminus of the cleavable peptide sequence. In certain embodiments, the linker group is glycine/serine rich (e.g., SRGSGXSGGPW SEQ ID NOS 11 and 41, wherein “X” is the cleavable peptide sequence). In certain embodiments, the target protease inhibition polypeptide sensor is a modified β-lactamase as described herein. For example, in certain embodiments, the target protease inhibition polypeptide sensor comprises SEQ ID NOS 30 and 42, wherein “X” is any one of SEQ ID NOs:31-32.

Certain embodiments of the invention provide an isolated nucleic acid encoding a target protease inhibition polypeptide sensor as described herein. In certain embodiments, the nucleic acid further comprises a promoter.

Certain embodiments of the invention provide an expression cassette comprising a nucleic acid as described herein and a promoter.

Certain embodiments of the invention provide a vector (e.g., a plasmid) comprising a nucleic acid or an expression cassette as described herein.

Certain embodiments of the invention provide a cell comprising a nucleic acid, expression cassette or vector as described herein.

The invention will now be illustrated by the following non-limiting Examples.

### Example 1. Functional Selection of Protease Inhibitory Antibodies

Proteases precisely control a wide variety of physiological processes and thus are important drug targets. Compared to small molecule inhibitors, monoclonal antibodies (mAbs) are attractive as they provide needed specificity. However, finding inhibitory mAbs is often the bottleneck largely due to lack of a function-based selection method. We overcame this obstacle and successfully isolated mAbs that inhibited five therapeutic targets spanning four basic classes of proteases. Our mAb inhibitors are highly selective and deliver desired biochemical and biological functions including reduction of amyloid beta formation in vitro and pain relief effects in animal tests. The technique described here can be readily applied to many biomedically important proteases for biologic inhibitor discovery and engineering.

### Results

Design of Functional Selection for Protease Inhibitory mAbs.

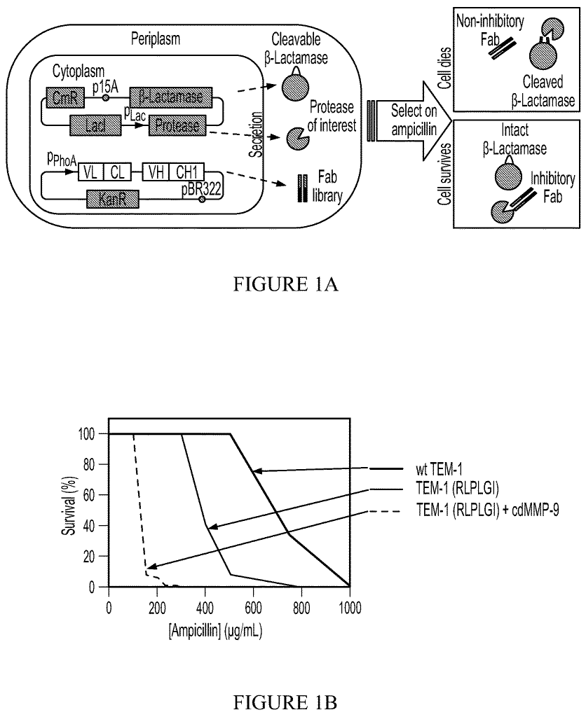

To select mAbs that inhibit proteases, three recombinant proteins—a clone from an antibody library, the protease of interest, and the protease substrate acting as an in vivo sensor—must be produced in the same location. We hypothesize that E. coli periplasmic co-expression is ideal for this task because the oxidative environment and associated molecular chaperons facilitate disulfide formation needed to produce antibody fragments and many human proteases in their active form. In addition, large combinatorial libraries have been routinely constructed in E. coli thanks to its high transformation efficiency. An important aspect of this method is a cellular protease inhibition sensor—our design is to engineer β-lactamase TEM-1, a periplasmic hydrolase of β-lactam antibiotics, by inserting a protease specific cleavable peptide sequence. When the modified TEM-1 is cleaved by the protease of interest, it will lose its β-lactam hydrolytic activity, and thus the cell cannot grow in the presence of ampicillin. Conversely, when proteolytic activity of the target is blocked by a co-expressed antibody, TEM-1 will be spared to confer ampicillin resistance to the host cell. Therefore, this live or die selection can identify antibody clones that specifically inhibit the activity of the targeted protease (FIG. 1A).

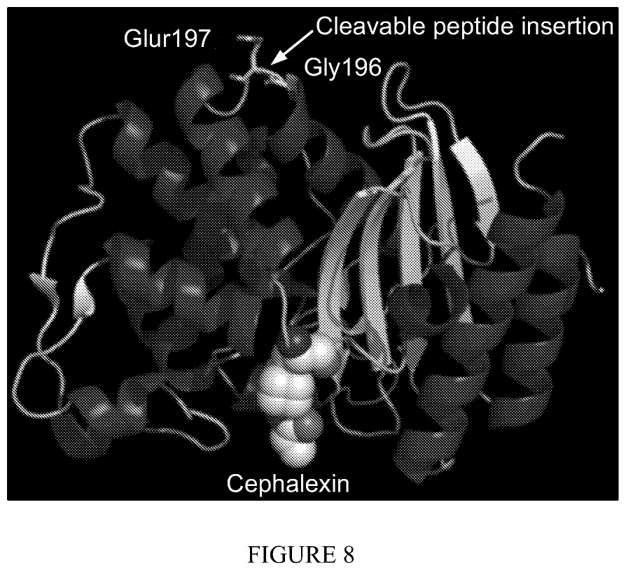

To demonstrate the generality of this functional selection method, matrix metalloproteinase-9 (MMP-9, neuropathic pain) (Kawasaki et al., Nat Med. 14, 331-316 (2008)) was evaluated. The extracellular/catalytic domain (cd) of this target, without the propeptide sequence, was cloned downstream of a pLac promoter and a pelB leader for periplasmic expression. Enzymatic assays showed that the produced protease was functional with expected activity (FIG. 7). A yield of 0.5-2.0 mg active soluble protease per liter of culture was typically achieved, suggesting the feasibility of its inhibition by co-expressed Fabs, which are usually produced at similar level in periplasm (Nam D H, Ge X. Biotechnol. Bioeng. 113, 717-723 (2016)).

### Distinct Selection Windows for Protease Inhibitors.

To select inhibitory mAbs with high potencies, a protease specific substrate with relatively fast kinetics (k_{cat}/Kₘs) was used for TEM-1 insertion sequence design. Specifically, synthetic peptide substrate RLPLGI (SEQ ID NO:31) was chosen for cdMMP-9. Flanked by flexible serine-glycine linkers at both ends, i.e. GSG-peptide-SGG, this cleavable peptide sequence was introduced between Gly196 and Glu197 of TEM-1 (FIG. 8). This site is located on an exposed surface loop away from the β-lactamase active center and has been exploited for the construction of cellular sensors (Galarneau et al., Nat Biotechnol. 20, 619-622 (2002); Porter et al., (2007) Anal Chem. 79:6702-6708). Survival rates of E. coli cells transformed with modified TEM-1 were measured on agar plates supplemented with 0-1000 μg/mL ampicillin. Results showed that the minimal inhibitory concentration (MICs) was 500 μg/mL or higher (FIG. 1B), suggesting that peptide insertion did not disrupt TEM-1 catalytic function nor was cleaved by endogenous proteases of E. coli periplasm. However, when cdMMP-9 was periplasmically co-expressed, MIC dramatically decreased to 200 μg/mL or lower, indicating that proteolytic cleavage of modified TEM-1 resulted in loss of their β-lactamase activity. Overall, the significant disparity between the survival curves with (dashed line in FIG. 1B) and without the co-expressed protease (solid lines in FIG. 1B) provided distinct windows for effective selection of inhibitors. Protease expression level was also up- or down-regulated by adding IPTG or glucose respectively to optimize selection conditions (Table 2). Specifically, 300 μg/mL ampicillin with 0.1 mM IPTG was determined. These conditions yielded a 100% survival rate in the absence of protease while nearly complete cell death (survival rates <10⁻⁶) in the presence of protease.

### Isolation of Multiple Potent mAb Inhibitors for Each Protease.

To promote generation of inhibitory mAbs targeting protease reaction clefts (Nam et al., Proc Natl Acad Sci USA 113, 14970-14975 (2016)), a human Fab synthetic library encoding CDR-H3s with 23-27 amino acids was constructed downstream of a phoA promoter and a STII leader (FIG. 1A). In the absence of ampicillin, transformation of obtained Fab library plasmids (1.1×10⁹ diversity) to E. coli competent cells carrying genes of modified TEM-1 and associated protease generated 1.5-8.6×10⁸ clones. Fab libraries were subjected to functional selection for each protease inhibition under pre-determined conditions (Table 2). Surviving colonies were then individually screened by culturing in liquid media under more stringent conditions, i.e. by increasing ampicillin concentration 100 μg/mL. After the initial selection, surviving clones were subjected to secondary screening. Among them, a percentage was randomly picked for DNA sequencing, and unique Fabs were identified (Table 2).

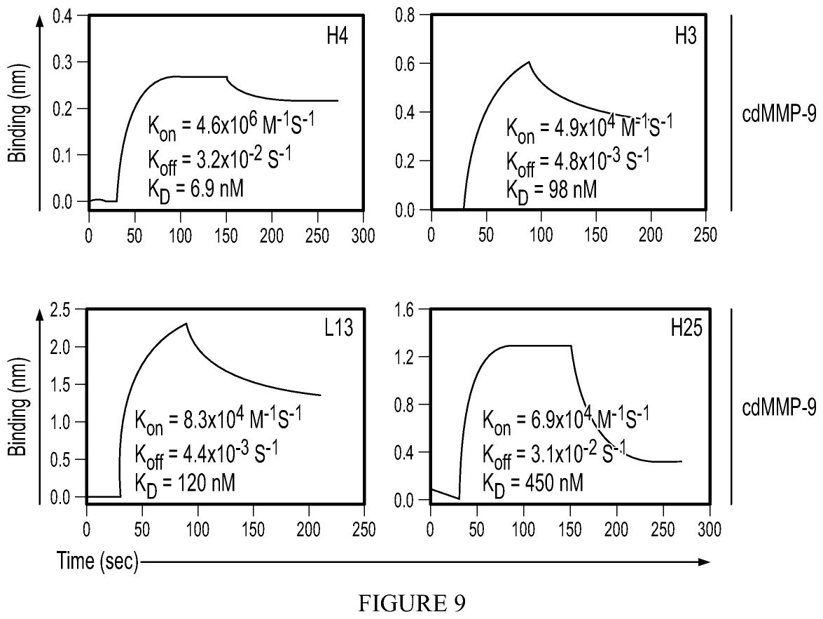

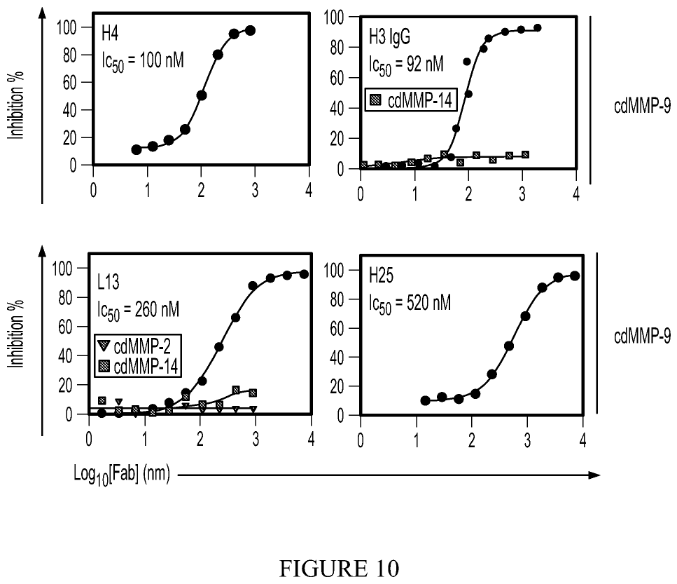

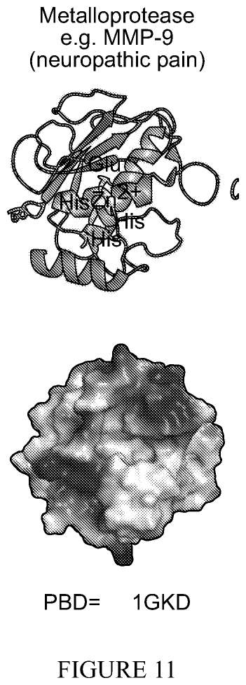

Four Fabs from were produced for biochemical characterizations (Table 1). Binding affinity measurements by biolayer interferometry and ELISA confirmed that all the tested Fabs bound to the protease target with dissociation constants (K_{D}s) ranging from less than 10 nM to more than 400 nM (FIG. 9). Among the Fabs tested, 3 of them had K_{D} values <250 nM. Particularly, anti-MMP9 Fab H4 exhibited a K_{D} of 6.9. Inhibitory function of purified Fabs was assayed with the protease and the FRET peptide substrate. Results indicated that most of the tested Fabs were inhibitors (FIG. 10). Among isolated inhibitory Fabs, 3 Fabs showed potent inhibition with calculated inhibition constant (K₁) values <250 nM (Table 1). Particularly, Fab H4 had a K₁ of 56 nM. Converting two anti-MMP9 inhibitory Fabs of nanomolar potencies into their IgG format increased the affinities and potencies as expected (Table 1). Rapid isolation of multiple potent inhibitory mAbs targeting all five tested proteases from >10⁸ library clones demonstrated the effectiveness and robustness of this selection system.

Inhibitory mAbs are Highly Selective, Functional on Physiological Substrates, and Proteolytically Stable.

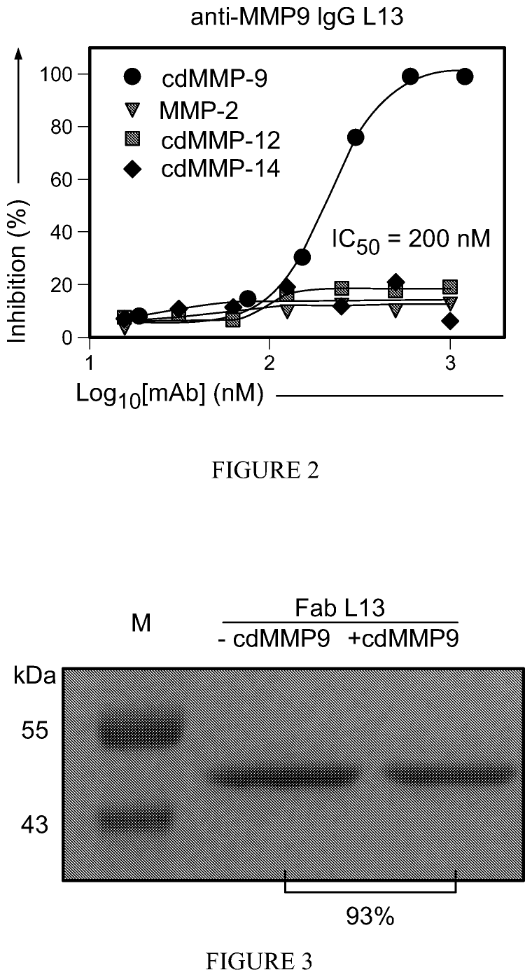

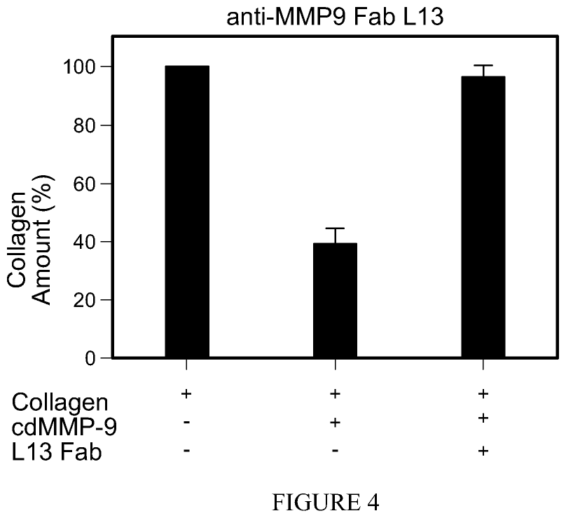

Inhibition selectivity of representative mAbs was tested. As results shown in FIG. 2 and FIG. 10, IgGs H3 and L13 inhibited cdMMP-9 with K₁s of 51 and 110 nM respectively but did not inhibit (cd)MMP-2/-12/-14 even at 1 μM. Next, anti-cdMMP-9 mAbs were examined for their inhibitory function on its physiological/macromolecular substrate. Under tested conditions, Fab L13 reduced the degradation of type I collagen by cdMMP-9 from 61% to 4% (FIG. 4). To test proteolytic stability of inhibitory antibodies in vitro, 1 μM purified Fabs were incubated with 1 μM protease at 37° C. SDS-PAGE revealed that after exposed to equal molar of the protease for 24 hours, Fab L13 remained 93% intact (FIG. 3).

### Identification of Active Site and Exosite Inhibitors.

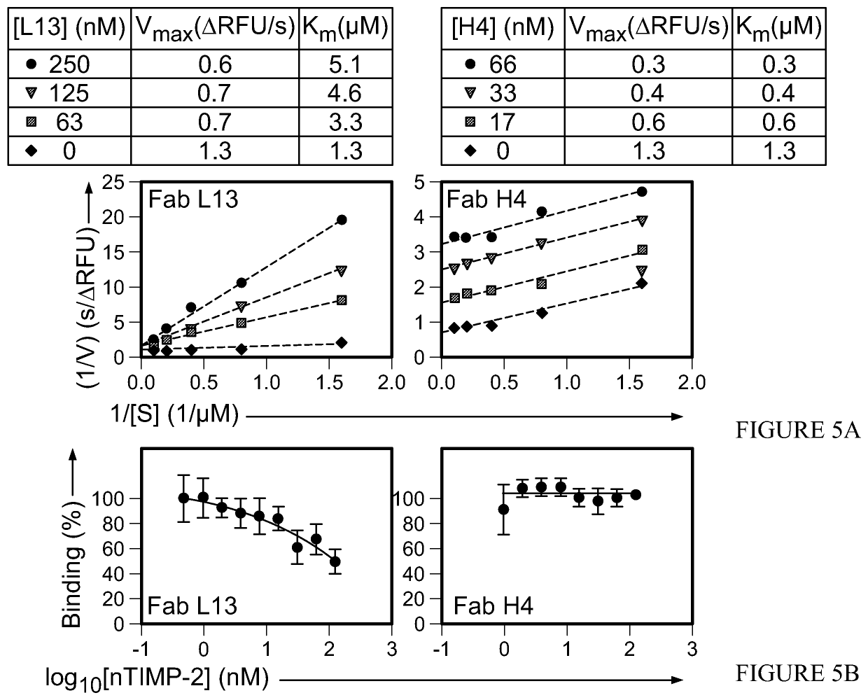

To understand the inhibition mechanism, we measured the protease kinetics in the presence of various concentrations of inhibitory mAbs. When concentration of Fab L13 increased from 0 to 250 nM, the Lineweaver-Burke plots of cdMMP-9 indicated an unaltered maximum velocity Vₘₐₓ and increased Michaelis constant Kₘ, suggesting that Fab L13 performed as a competitive inhibitor (FIG. 5A). In contrast, with increasing concentrations of Fab H4, the kinetics of cdMMP-9 showed decreases on both Vₘₐₓ and Kₘ values, suggesting an uncompetitive inhibition mode. In addition, ELISA of Fab L13 on immobilized cdMMP-9 with the presence of nTIMP-2, an endogenous inhibitor of MMP-9 recognizing its reaction cleft, revealed that high concentrations of nTIMP-2 displaced L13 on binding to cdMMP-9 (FIG. 5B). However, nTIMP-2 did not interfere H4 on its binding to cdMMP-9 in a similar competitive ELISA. Collectively, these results imply that Fab L13 is an active site competitive inhibitor, and Fab H4 is an exosite uncompetitive inhibitor presumably delivering its blockage function by an allosteric mechanism.

### Anti-MMP9 IgG L13 Exhibits Pain Attenuation Efficacy In Vivo.

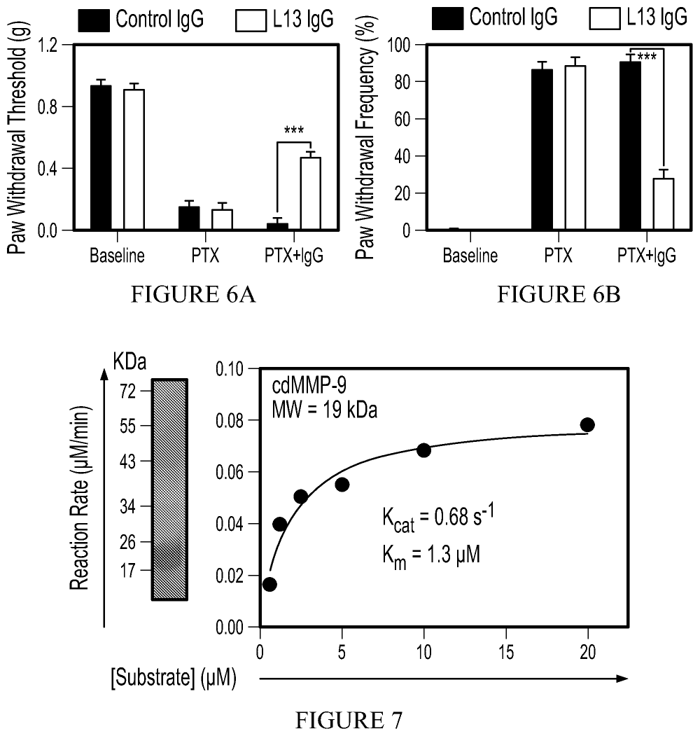

As MMP-9 is required in the early phase of neuropathic pain development after nerve injury (Kawasaki et al., Nat Med. 14, 331-316 (2008)), we further evaluated the pain relief efficacy of MMP-9 inhibitory IgG L13 in paclitaxel (PTX)-induced neuropathic pain in mice. PTX evoked robust mechanical allodynia, a cardinal feature of neuropathic pain, by decreasing paw withdrawal threshold (FIG. 6A) and increasing paw withdrawal frequency to a subthreshold filament (0.6 g, FIG. 6B). In contrast to polyclonal human control IgG which did not change the animals' behavioral responses, intrathecal injection of 200 μg IgG L13, given 15 days after the first PTX administration, significantly increased the threshold and reduced the withdrawal frequency (p<0.001, two-way ANOVA) (FIG. 6).

### DISCUSSION

In this study, we evaluated an inhibitory antibody discovery method using MMP-9 as an example protease. This functional selection method achieved an exceptionally high success rate. However, some false positive clones were identified. We further analyzed these clones, aiming to understand how they could survive during this live or die selection. Two non-inhibitory anti-MMP9 Fabs were isolated when SGRIGFLRTA (SEQ ID NO:32), an MMP generic substrate with relatively slow kinetics on MMP-9, was used for TEM-1 insertion. It is possible that inefficient hydrolysis of TEM-1 spares its β-lactamase activity, which otherwise should be fully abolished by a complete protease cleavage. In contrast, when a fast substrate RLPLGI (SEQ ID NO:31) was used, all eight isolated anti-MMP9 Fabs were inhibitors (Table 2). Thus, choosing peptide inserts with fast cleavage kinetics is important for efficient selection of protease inhibitory mAbs.

Other than antibody library and peptide insertion sequence designs, the selection conditions, such as concentrations of ampicillin and inducer, culture media, and temperature, can also be customized for a particular protease target, allowing rapid downsizing of libraries. Our selection resulted in potent mAbs—more than half of identified inhibitors showed a K₁<250 nM. Some isolated inhibitors however had weak potency (K₁>1 μM), presumably caused by a high expression level of certain Fabs which compensates their low potency. In addition, our approach of periplasmic co-expression facilitates the disulfide formation required for activities of many human proteases. Furthermore, proteases were produced in their propeptide-free form, thus isolated mAbs can directly inhibit the activated proteases. Certain macromolecular inhibitors of proteases, especially the canonical mechanism inhibitors including endogenous inhibitors and inhibitory mAbs, tend to be slowly cleaved by the targeted protease (Farady C J, Craik C S. (2010) Chembiochem. 11:2341-2346). However, the mAbs isolated in this study exhibited excellent proteolytic stability (FIG. 3). Likely it was benefited from the nature of in vivo selection, because inhibitory function and thus the integrity of Fabs must be maintained over the entire course for cell survival.

In summary, this study described a high-throughput method for selecting protease inhibitory mAbs. Compared to recent technology developments such as epitope synthetic mimicry (Sela-Passwell, et al., Nat Med. 18, 143-147 (2012)), convex paratope design (Nam et al., Proc Natl Acad Sci USA 113, 14970-14975 (2016)), competitive phage elution (Devy et al., Cancer Res. 69, 1517-1526 (2009)), cytoplasmic genetic selection (Gal-Tanamy et al., J Mol Biol. 347, 991-1003 (2005)), and epitope-specific FACS (Lopez et al., Biotechnol Bioeng. 115, 2673-2682 (2018)), this method directly relies on functional inhibition and offers the following advantages: (i) an exceptionally high successful rate as ratio of inhibitors over binders (Table 2); (ii) exclusive selectivity against proteases of the same family (FIG. 2 & FIG. 10); (iii) isolated mAbs are proteolytic stable and cannot be cleaved by targeted proteases (FIG. 3); and (iv) both active-site and allosteric inhibitors can be identified (FIG. 5). Overall, the method demonstrated here can be readily applied for the development of inhibitory mAbs targeting a large variety of biomedically important proteases. In addition, since natural inhibitors of proteases are often characterized by broad selectivity, our study can also be used for engineering endogenous inhibitors with improved specificity.

### Materials and Methods

### Development of Protease Cleavage Reporters

Plasmid carrying β-lactamase TEM-1 gene was PCR amplified to introduce unique XbaI and NcoI recognition sites between G196 and E197 of TEM-1. The PCR product was ligated with 5′ phosphorylated oligonucleotide assembled adapters encoding protease specific cleavable peptide sequences (Table 2) flanked by serine-glycine linkers (GSG[peptide]SGG) to obtain modified TEM-1s. The gene encoding the catalytic domain of human MMP-9 (residue 107-443 without fibronectin domains), without its associated propeptide sequence, was PCR assembled and cloned into SfI sites on pMopac16 carrying a p15A origin and a pelB leader peptide to obtain a periplasmic expression plasmid (Nam D H, Ge X. Biotechnol. Bioeng. 113, 717-723 (2016)). The modified TEM-1 gene was then sub-cloned into the protease expression plasmid using NsiI and NheI sites to generate a reporter plasmid (FIG. 1A). The cloned plasmid was confirmed by DNA sequencing. β-lactam ring hydrolysis activities of modified TEM-1s in the absence or presence of protease were assayed by culturing transformed E. coli BL21 cells at serial dilutions on 2×YT agar plates containing 34 μg/mL chloramphenicol, 50 μg/mL kanamycin, 0-0.1 mM IPTG, 0-2% glucose, and 0-1000 μg/mL ampicillin at 30° C. for 16 hours. The ratios of colony numbers on ampicillin plates over colony numbers on ampicillin-free plates were calculated as survival rates, which were used to identify the optimal conditions of inhibitor selection for the protease target.

### Selection of Protease Inhibitory Antibodies

Fab library genes containing regular length (Persson et al., J Mol Biol. 425, 803-811 (2013)) or ultra-long CDR-H3s (Nam D H, Ge X. et al., Methods Mol Biol. 1731, 307-324 (2018)) were PCR amplified and cloned into pHPK (kanR, pBR322, phoA promoter, and STII leader peptide). Constructed library plasmids pHPK-Fab were transformed into E. coli Jude-I electrocompetent cells for amplification. Randomly picked colonies were sequenced for library quality and diversity tests. Electrocompetent cells of BL21 harboring the reporter plasmid for individual protease were transformed with 100 μg library pHPK-Fab. Transformed cells were cultured on 2×YT agar plates of pre-determined selection conditions specific for MMP-9 (Table 2). Small aliquots of transformed cells were also serially diluted and cultured on 2×YT agar plates supplemented with 34 μg/mL chloramphenicol and 50 μg/mL kanamycin for library size determination. Colonies surviving the initial selection were individually inoculated in the 2×YT selection media with a higher ampicillin concentration for secondary screening. Well-grown clones were selected for Fab plasmid extraction and V_{H} and V_{L} DNA sequencing.

### Production of Antibodies and Proteases

Fab expression plasmids of isolated antibodies were transformed into BL21 cells for periplasmic production by culturing in 2×YT media at 30° C. for 12 hours. Fabs with a hexahistidine tag (SEQ ID NO: 39) at the C-terminal of V_{H} were purified using Ni-NTA agarose (Qiagen) from periplasmic fractions prepared by lysozyme and osmotic shock (Rodriguez et al., Appl Biochem Biotechnol. 183, 520-529 (2017)). Associated IgGs were produced in HEK293F (ThermoFisher Scientific) as previously described (Chen et al., Oncotarget. 9, 29431-29444 (2018)). Purified Fabs and IgGs were dialyzed at 4° C. against the following assay buffers: 50 mM Tris-HCl pH 7.5, 150 mM NaCl, 5 mM CaCl₂), 0.4 mM ZnCl₂ for cdMMP-9. Dialyzed antibody samples were concentrated by 10 kDa MWCO ultrafiltration (Amicon), and their purity and concentration were determined by SDS-PAGE and UV spectrophotometer (BioTek). C-terminal hexahistidine tagged (SEQ ID NO: 39) cdMMP-9, cdMMP-12 and cdMMP-14 were produced in their active format in the periplasmic space of E. coli without refolding or activation (Nam D H, Ge X. Biotechnol. Bioeng. 113, 717-723 (2016)) and purified using Ni-NTA agarose (Qiagen). MMP-2 was purchased from AnaSpec Inc. Cultured media was clarified by centrifugation and 0.45 μm filtration.

### Biochemical Characterizations of Isolated Antibodies

Binding kinetics of produced antibodies towards MMP-9 were analyzed by using biolayer interferometry (ForteBio). For Fabs, biotinylated proteases were immobilized on streptavidin biosensors, and Fab binding to the sensors in absence of protease was monitored as backgrounds. For IgGs, protein A sensors were used and protease bindings without IgG were checked as backgrounds. kₒₙ and k_{off} were determined for K_{D} calculations. Competitive ELISA of Fabs on immobilized cdMMP-9/-14 in the presence of 1 nM-1 μM nTIMP-2 was also tested. Fab in vitro stability was tested by incubating 1 μM Fab with 1 μM of the respective protease in the assay buffer for 12 hours and the samples were analyzed by SDS-PAGE.

For inhibition tests, 1 μM Fabs were 2-fold serially diluted into protease specific assay buffer and incubated with 1-10 nM proteases for 30 min at room temperature. The kinetic measurements were started with the addition of 1 μM following FRET peptide substrates: M-2350 (Bachem) for MMP-9/14. The generated fluorescence signals were monitored with excitation and emission wavelengths at 325 and 392 nm (except M-2595 at 320/420 nm) using a fluorescence plate reader (BioTek). Inhibition percentages at given concentrations were calculated by comparing the initial reaction rates in the presence or absence of inhibitor. IC₅₀ was determined as the concentration that achieved 50% inhibition, and K₁ values were calculated using K₁=IC₅₀/(S/Kₘ+1). Vₘₐₓ and Kₘ at various Fab concentrations were measured to determine the inhibition type. FRET inhibition assays were also used for selectivity tests of isolated Fabs with the relevant proteases.

For inhibition tests on macromolecular substrates, 1 μM cdMMP-9 was incubated with 300 μg/mL rat collagen I (Corning) with or without 1 μM Fab L13 in MMP-9 assay buffer at 37° C. for 24 h. Samples were taken hourly and analyzed by SDS-PAGE under non-reducing conditions.

### Biological Functions of Anti-MMP9 IgG L13: Neuropathic Pain Measurement.

Wild-type CD1 mice (male and female, 8-10 wks, Charles River Laboratories) were housed at Duke vivarium animal facility, and all animal experiment protocols were approved. To produce chemotherapy-associated neuropathic pain, paclitaxel (PTX, 2 mg/kg, i.p.) was injected at day 0, 2, 4, and 6 (Chen et al., J Clin Invest. 125, 3226-3240 (2015)). Intrathecal injection was performed as described previously (Xu et al., Nat Med. 21, 1326-1331 (2015)), mice were anesthetized with isoflurane and a spinal cord puncture was performed between the L5 and L6 level to deliver drugs (10 μL) using a 30G needle. All behavioral tests were performed in boxes on an elevated metal mesh floor under stable room temperature and humidity. Mice were habituated to the environment for at least 2 days before the experiments. To assess mechanical allodynia, the plantar surface of the left hind-paw was stimulated using a series of von Frey fibers with logarithmically increasing stiffness (0.02-2.56 gram, Stoelting), presented perpendicularly to the central plantar surface. 50% paw withdrawal threshold was determined following Dixon's up-down method. The frequency response was measured by stimulating the hind-paw with a 0.4 gram von Frey hair for ten times and the percentage withdrawal response was calculated as frequency. All the behavioral tests were performed in a blinded manner.

### Tables

TABLE 1
Isolated inhibitory antibodies toward MMP-9.

| | Cleavable | | | Binding | Inhibition |
|---|---|---|---|---|---|
| Target | Peptide | | | Affinity | Potency K₁ |
| (indication) | on TEM-1 | Fab* | CDR-H3 (length) | K_{D} (nM) | (nM)^{†} |
| MMP-9 | RLPLGI | H4 | SSLAWAQDRVYKPVEAMTWAYGMDY | 6.9 | 56 |
| (neuropathic | (SEQ ID | | (25) (SEQ ID NO: 8) | | |
| pain) | NO: 31) | | | | |
| | | H3 | RFEPGLLKRNKRWISYTLCEAGYGM | 98 (71)^{‡} | 66 (51)^{‡} |
| | | | DY (27) (SEQ ID NO: 17) | | |
| | | L13 | KYMVFGTRMGWVEHTDFAGQGYYAM | 120 (53)^{‡} | 150 (110)^{‡} |
| | | | DY (27) (SEQ ID NO: 25) | | |
| | | H25 | CKLYTSYMIPVGSDSVNRCMSSYGM | 450 | 290 |
| | | | DY (27) (SEQ ID NO: 28) | | |

Note:
*Only Fabs with inhibition constants (K₁s) < 500 nM are shown,
^{†}Clones are ranked by their K₁ values.
^{‡}Data of associated IgGs are shown in parentheses.

TABLE 2
Conditions and statistics of selections for protease inhibitory antibodies

| | cdMMP-9 | |
|---|---|---|
| Protease | RLPLGI | SGRIGFLRTA |
| Peptide insert | (SEQ ID NO:31) | (SEQ ID NO:32) |
| Library | | |
| CDR-H3 length (aa) | 23, 25, 27 | 23, 25, 27 |
| Size | 6.2 × 10⁸ | 4.1 × 10⁸ |
| Initial selection | | |
| [Amp] (μg/mL) | 300 | 300 |
| [IPTG] (mM) | 0.1 | 0.1 |
| [glucose] (%) | 0 | 0 |
| Temp (° C.) | 30 | 30 |
| # of clones remaining | 22 | 37 |
| Secondary screening | | |
| [Amp] (μg/mL) | 400 | 400 |
| [IPTG] (mM) | 0.1 | 0.1 |
| [glucose] (%) | 0 | 0 |
| Temp (° C.) | 30 | 30 |
| # of clones remaining | 13 | 15 |
| Sequenced | 13 | 7 |
| Unique correct sequences | 13 | 7 |
| Fabs produced | 8 | 5 |
| Binders | 8 | 5 |
| Inhibitors | 8 | 3 |

Note:
*Long CDR library (CDR-H3 length = 23, 25 or 27) and normal CDR library (CDR-H3 length = 5-21) were combined for selection.
†Only 10 clones were randomly picked for secondary screening.

TABLE 3
Clone Sequences

| SEQ ID | | |
|---|---|---|
| NO(S): | SEQUENCE | DESCRIPTION |
| 1 | DIQMTQSPSSLSASVGDRVTITCRASQSVSSAVAWYQQKPGKAPKL | Clone H4: light chain |
| | LIYSASSLYSGVPSRFSGSRSGTDFTLTISSLQPEDFATYYCQQSY | variable region; CDRs |
| | HPLFTFGQGTKVEIKR | bolded. |
| 2 | RASQSVSSAVA | Clone H4: CDR-L1 |
| 3 | SASSLYS | Clone H4: CDR-L2 |
| 4 | QQSYHPLFT | Clone H4: CDR-L3 |
| 5 | EVQLVESGGGLVQPGGSLRLSCAASGFNIYSYSIHWVRQAPGKGLE | Clone H4: heavy chain |
| | WVAYIYPSSGYTYYADSVKGRFTISADTSKNTAYLQMNSLRAEDTA | variable region; CDRs |
| | VYYCARSSLAWAQDRVYKPVEAMTWAYGMDYWGQGTLVTVSSAS | bolded. |
| 6 | GFNIYSYSIH | Clone H4: CDR-H1 |
| 7 | YIYPSSGYTYYADSVK | Clone H4: CDR-H2 |
| 8 | SSLAWAQDRVYKPVEAMTWAYGMDY | Clone H4: CDR-H3 |
| 9 | DIQMTQSPSSLSASVGDRVTITCRASQSVSSAVAWYQQKPGKAPKL | Clone H3: light chain |
| | LIYSASSLYSGVPSRFSGSRSGTDFTLTISSLQPEDFATYYCQQAS | variable region; CDRs |
| | HLITFGQGTKVEIKR | bolded. |
| 2 | RASQSVSSAVA | Clone H3: CDR-L1 |
| 3 | SASSLYS | Clone H3: CDR-L2 |
| 12 | QQASHLIT | Clone H3: CDR-L3 |
| 13 | EVQLVESGGGLVQPGGSLRLSCAASGFNIYSSSMHWVRQAPGKGLE | Clone H3: heavy chain |
| | WVAYIYSSYGYTYYADSVKGRFTISADTSKNTAYLQMNSLRAEDTA | variable region; CDRs |
| | VYYCARRFEPGLLKRNKRWISYTLCEAGYGMDYWGQGTLVTVSSAS | bolded. |
| 15 | GFNIYSSSMH | Clone H3: CDR-H1 |
| 16 | YIYSSYGYTYYADSVK | Clone H3: CDR-H2 |
| 17 | RFEPGLLKRNKRWISYTLCEAGYGMDY | Clone H3: CDR-H3 |
| 18 | DIQMTQSPSSLSASVGDRVTITCRASQSVSSAVAWYQQKPGKAPKL | Clone L13: light chain |
| | LIYSASSLYSGVPSRFSGSRSGTDFTLTISSLQPEDFATYYCQQYA | variable region; CDRs |
| | ALITFGQGTKVEIKR | bolded. |
| 2 | RASQSVSSAVA | Clone L13: CDR-L1 |
| 3 | SASSLYS | Clone L13: CDR-L2 |
| 20 | QQYAALIT | Clone L13: CDR-L3 |
| 21 | EVQLVESGGGLVQPGGSLRLSCAASGFNISSSYIHWVRQAPGKGLE | Clone L13: heavy chain |
| | WVASISSSSGYTSYADSVKGRFTISADTSKNTAYLQMNSLRAEDTA | variable region; CDRs |
| | VYYCARKYMVFGTRMGWVEHTDFAGQGYYAMDYWGQGTLVTVSSAS | bolded. |
| 23 | GFNISSSYIH | Clone L13: CDR-H1 |
| 24 | SISSSSGYTSYADSVK | Clone L13: CDR-H2 |
| 25 | KYMVFGTRMGWVEHTDFAGQGYYAMDY | Clone L13: CDR-H3 |
| 37 | DIQMTQSPSSLSASVGDRVTITCRASQSVSSAVAWYQQKPGKAPKL | Clone H25: light chain |
| | LIYSASSLYSGVPSRFSGSRSGTDFTLTISSLQPEDFATYYCQQGG | variable region; CDRs |
| | GASLITFGQGTKVEIKR | bolded. |
| 2 | RASQSVSSAVA | Clone H25: CDR-L1 |
| 3 | SASSLYS | Clone H25: CDR-L2 |
| 38 | QQGGGASLIT | Clone H25: CDR-L3 |
| 26 | EVQLVESGGGLVQPGGSLRLSCAASGFNIYSSSMHWVRQAPGKGLE | Clone H25: heavy chain |
| | WVASIYSYYGYTYYADSVKGRFTISADTSKNTAYLQMNSLRAEDTA | variable region; CDRs |
| | VYYCARCKLYTSYMIPVGSDSVNRCMSSYGMDYWGQGTLVTVSSAS | bolded. |
| 15 | GFNIYSSSMH | Clone H25: CDR-H1 |
| 27 | SIYSYYGYTYYADSVK | Clone H25: CDR-H2 |
| 28 | CKLYTSYMIPVGSDSVNRCMSSYGMDY | Clone H25: CDR-H3 |
| 29 | MSIQHFRVALIPFFAAFCLPVFAHPETLVKVKDAEDQLGARVGYIE | TEM-1 Sequence |
| | LDLNSGKILESFRPEERFPMMSTFKVLLCGAVLSRVDAGQEQLGRR | |
| | IHYSQNDLVEYSPVTEKHLTDGMTVRELCSAAITMSDNTAANLLLT | |
| | TIGGPKELTAFLHNMGDHVTRLDRWEPELNEAIPNDERDTTMPAAM | |
| | ATTLRKLLTGELLTLASRQQLIDWMEADKVAGPLLRSALPAGWFIA | |
| | DKSGAGERGSRGIIAALGPDGKPSRIVVIYTTGSQATMDERNRQIA | |
| | EIGASLIKHW | |
| 30 and | MSIQHFRVALIPFFAAFCLPVFAHPETLVKVKDAEDQLGARVGYIE | Modified TEM-1 Sequence. |
| 42 | LDLNSGKILESFRPEERFPMMSTFKVLLCGAVLSRIDAGQEQLGRR | “X” represents the |
| | IHYSQNDLVEYSPVTEKHLTDGMTVRELCSAAITMSDNTAANLLLT | cleavable peptide insert. |
| | TIGGPKELTAFLHNMGDHVTRLDRWEPELNEAIPNDERDTTMPVAM | |
| | ATTLRKLLTGSRGSGXSGGPWELLTLASRQQLIDWMEADKVAGPLL | |
| | RSALPAGWFIADKSGAGERGSRGIIAALGPDGKPSRIVVIYTTGSQ | |
| | ATMDERNRQIAEIGASLIKHW | |
| 31 | RLPLGI | MMP-9 Cleavable Peptide |
| | | Insert |
| 32 | SGRIGFLRTA | MMP Generic Cleavable |
| | | Peptide Insert |
| 11 and | SRGSGXSGGPW | Cleavable Peptide Insert |
| 41 | | with linking groups at |
| | | the N′ and C′ terminal |
| | | ends; “X” represents the |
| | | cleavable peptide insert |

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. An isolated anti-matrix metalloproteinase-9 (MMP-9) antibody or fragment thereof, comprising one or more complementarity determining regions (CDRs) selected from the group consisting of:
(a) a light chain CDR1 having at least 80% sequence identity to an amino acid sequence of RASQSVSSAVA (SEQ ID NO:2);
(b) a light chain CDR2 having at least 80% sequence identity to an amino acid sequence of SASSLYS (SEQ ID NO:3);
(c) a light chain CDR3 having at least 80% sequence identity to an amino acid sequence of any one of QQYAALIT (SEQ ID NO:20), QQSYHPLFT (SEQ ID NO:4), QQASHLIT (SEQ ID NO:12) and QQGGGASLIT (SEQ ID NO:38);
(d) a heavy chain CDR1 having at least 80% sequence identity to an amino acid sequence of any one of GFNISSSYIH (SEQ ID NO:23), GFNIYSYSIH (SEQ ID NO:6), and GFNIYSSSMH (SEQ ID NO:15);
(e) a heavy chain CDR2 having at least 80% sequence identity to an amino acid sequence of any one of SISSSSGYTSYADSVK (SEQ ID NO:24), YIYPSSGYTYYADSVK (SEQ ID NO:7), YIYSSYGYTYYADSVK (SEQ ID NO:16), and SIYSYYGYTYYADSVK (SEQ ID NO:27); and
(f) a heavy chain CDR3 having at least 80% sequence identity to an amino acid sequence of any one of KYMVFGTRMGWVEHTDFAGQGYYAMDY (SEQ ID NO:25), SSLAWAQDRVYKPVEAMTWAYGMDY (SEQ ID NO:8), RFEPGLLKRNKRWISYTLCEAGYGMDY (SEQ ID NO:17), and CKLYTSYMIPVGSDSVNRCMSSYGMDY (SEQ ID NO:28).

2. (canceled)

3. The isolated anti-MMP-9 antibody of claim 1, or fragment thereof, comprising:
(a) a light chain CDR1 comprising the amino acid sequence of SEQ ID NO:2;
(b) a light chain CDR2 comprising the amino acid sequence of SEQ ID NO:3;
(c) a light chain CDR3 comprising the amino acid sequence of any one of SEQ ID NOs:20, 4, 12, and 38;
(d) a heavy chain CDR1 comprising the amino acid sequence of any one of SEQ ID NOs:23, 6, and 15;
(e) a heavy chain CDR2 comprising the amino acid sequence of any one of SEQ ID NOs:24, 7, 16, and 27; and
(f) a heavy chain CDR3 comprising the amino acid sequence of any one of SEQ ID NOs:25, 8, 17, and 28.

4. The isolated anti-MMP-9 antibody of claim 1, or fragment thereof, comprising:
(a) a light chain CDR1 comprising the amino acid sequence of SEQ ID NO:2;
(b) a light chain CDR2 comprising the amino acid sequence of SEQ ID NO: 3; and
(c) a light chain CDR3 comprising the amino acid sequence of SEQ ID NO:20;
and/or
(d) a heavy chain CDR1 comprising the amino acid sequence of SEQ ID NO:23;
(e) a heavy chain CDR2 comprising the amino acid sequence of SEQ ID NO:24; and
(f) a heavy chain CDR3 comprising the amino acid sequence of SEQ ID NO:25.

5. (canceled)

6. The isolated anti-MMP-9 antibody of claim 4, or fragment thereof, comprising a light chain CDR1 comprising the amino acid sequence of SEQ ID NO:2, a light chain CDR2 comprising the amino acid sequence of SEQ ID NO:3, a light chain CDR3 comprising the amino acid sequence of SEQ ID NO:20, a heavy chain CDR1 comprising the amino acid sequence of SEQ ID NO:23, a heavy chain CDR2 comprising the amino acid sequence of SEQ ID NO:24, and a heavy chain CDR3 comprising the amino acid sequence of SEQ ID NO:25.

7. The isolated anti-MMP-9 antibody of claim 1, or fragment thereof, comprising:
(a) a light chain CDR1 comprising the amino acid sequence of SEQ ID NO:2;
(b) a light chain CDR2 comprising the amino acid sequence of SEQ ID NO:3; and
(c) a light chain CDR3 comprising the amino acid sequence of SEQ ID NO:4;
and/or
(d) a heavy chain CDR1 comprising the amino acid sequence of SEQ ID NO:6;
(e) a heavy chain CDR2 comprising the amino acid sequence of SEQ ID NO:7; and
(f) a heavy chain CDR3 comprising the amino acid sequence of SEQ ID NO: 8.

8. (canceled)

9. The isolated anti-MMP-9 antibody of claim 7, or fragment thereof, comprising a light chain CDR1 comprising the amino acid sequence of SEQ ID NO:2, a light chain CDR2 comprising the amino acid sequence of SEQ ID NO:3, a light chain CDR3 comprising the amino acid sequence of SEQ ID NO:4, a heavy chain CDR1 comprising the amino acid sequence of SEQ ID NO:6, a heavy chain CDR2 comprising the amino acid sequence of SEQ ID NO:7, and a heavy chain CDR3 comprising the amino acid sequence of SEQ ID NO:8.

10. The isolated anti-MMP-9 antibody of claim 1, or fragment thereof, comprising:
(a) a light chain CDR1 comprising the amino acid sequence of SEQ ID NO:2;
(b) a light chain CDR2 comprising the amino acid sequence of SEQ ID NO: 3; and
(c) a light chain CDR3 comprising the amino acid sequence of SEQ ID NO: 12;
and/or
(d) a heavy chain CDR1 comprising the amino acid sequence of SEQ ID NO:15;
(e) a heavy chain CDR2 comprising the amino acid sequence of SEQ ID NO: 16; and
(f) a heavy chain CDR3 comprising the amino acid sequence of SEQ ID NO: 17.

11. (canceled)

12. The isolated anti-MMP-9 antibody of claim 10, or fragment thereof, comprising a light chain CDR1 comprising the amino acid sequence of SEQ ID NO:2, a light chain CDR2 comprising the amino acid sequence of SEQ ID NO:3, a light chain CDR3 comprising the amino acid sequence of SEQ ID NO:12, a heavy chain CDR1 comprising the amino acid sequence of SEQ ID NO:15, a heavy chain CDR2 comprising the amino acid sequence of SEQ ID NO:16, and a heavy chain CDR3 comprising the amino acid sequence of SEQ ID NO:17.

13. The isolated anti-MMP-9 antibody of claim 1, or fragment thereof, comprising:
(a) a light chain CDR1 comprising the amino acid sequence of SEQ ID NO:2;
(b) a light chain CDR2 comprising the amino acid sequence of SEQ ID NO: 3; and
(c) a light chain CDR3 comprising the amino acid sequence of SEQ ID NO: 38;
and/or
(d) a heavy chain CDR1 comprising the amino acid sequence of SEQ ID NO:15;
(e) a heavy chain CDR2 comprising the amino acid sequence of SEQ ID NO:27; and
(f) a heavy chain CDR3 comprising the amino acid sequence of SEQ ID NO:28.

14. (canceled)

15. The isolated anti-MMP-9 antibody of claim 13, or fragment thereof, comprising a light chain CDR1 comprising the amino acid sequence of SEQ ID NO:2, a light chain CDR2 comprising the amino acid sequence of SEQ ID NO:3, a light chain CDR3 comprising the amino acid sequence of SEQ ID NO:38, a heavy chain CDR1 comprising the amino acid sequence of SEQ ID NO:15, a heavy chain CDR2 comprising the amino acid sequence of SEQ ID NO:27, and a heavy chain CDR3 comprising the amino acid sequence of SEQ ID NO:28.

16. The isolated anti-MMP-9 antibody of claim 1, or fragment thereof, comprising:
1) a light chain variable region comprising an amino acid sequence that has at least 80% sequence identity to any one of:
(a)
(SEQ ID NO: 18)
DIQMTQSPSSLSASVGDRVTITCRASQSVSSAVAWYQQKPGKAPKLLIYS
ASSLYSGVPSRFSGSRSGTDFTLTISSLQPEDFATYYCQQYAALITFGQG
TKVEIKR;
(b)
(SEQ ID NO: 1)
DIQMTQSPSSLSASVGDRVTITCRASQSVSSAVAWYQQKPGKAPKLLIYS
ASSLYSGVPSRFSGSRSGTDFTLTISSLQPEDFATYYCQQSYHPLFTFGQ
GTKVEIKR;
(c)
(SEQ ID NO: 9)
DIQMTQSPSSLSASVGDRVTITCRASQSVSSAVAWYQQKPGKAPKLLIYS
ASSLYSGVPSRFSGSRSGTDFTLTISSLQPEDFATYYCQQASHLITFGQG
TKVEIKR;
and
(d)
(SEQ ID NO: 37)
DIQMTQSPSSLSASVGDRVTITCRASQSVSSAVAWYQQKPGKAPKLLIYS
ASSLYSGVPSRFSGSRSGTDFTLTISSLQPEDFATYYCQQGGGASLITFG
QGTKVEIKR;
and/or
2) a heavy chain variable region comprising an amino acid sequence that has at least 80% sequence identity to any one of:
(a)
(SEQ ID NO: 21)
EVQLVESGGGLVQPGGSLRLSCAASGFNISSSYIHWVRQAPGKGLEWVAS
ISSSSGYTSYADSVKGRFTISADTSKNTAYLOMNSLRAEDTAVYYCARKY
MVFGTRMGWVEHTDFAGQGYYAMDYWGQGTLVTVSSAS;
(b)
(SEQ ID NO: 5)
EVQLVESGGGLVQPGGSLRLSCAASGFNIYSYSIHWVRQAPGKGLEWVAY
IYPSSGYTYYADSVKGRFTISADTSKNTAYLQMNSLRAEDTAVYYCARSS
LAWAQDRVYKPVEAMTWAYGMDYWGQGTLVTVSSAS;
(c)
(SEQ ID NO: 13)
EVQLVESGGGLVQPGGSLRLSCAASGFNIYSSSMHWVRQAPGKGLEWVAY
IYSSYGYTYYADSVKGRFTISADTSKNTAYLOMNSLRAEDTAVYYCARRF
EPGLLKRNKRWISYTLCEAGYGMDYWGQGTLVTVSSAS;
and
(d)
(SEQ ID NO: 26)
EVQLVESGGGLVQPGGSLRLSCAASGFNIYSSSMHWVRQAPGKGLEWVAS
IYSYYGYTYYADSVKGRFTISADTSKNTAYLQMNSLRAEDTAVYYCARCK
LYTSYMIPVGSDSVNRCMSSYGMDYWGQGTLVTVSSAS.

17. (canceled)

18. The isolated anti-MMP-9 antibody of claim 1, or fragment thereof, comprising:
a light chain variable region comprising an amino acid sequence that has at least 90% sequence identity to SEQ ID NO:18 and a heavy chain variable region comprising an amino acid sequence that has at least 90% sequence identity to SEQ ID NO:21;
a light chain variable region comprising an amino acid sequence that has at least 90% sequence identity to SEQ ID NO:1 and a heavy chain variable region comprising an amino acid sequence that has at least 90% sequence identity to SEQ ID NO:5;
a light chain variable region comprising an amino acid sequence that has at least 90% sequence identity to SEQ ID NO:9 and a heavy chain variable region comprising an amino acid sequence that has at least 90% sequence identity to SEQ ID NO:13; or
a light chain variable region comprising an amino acid sequence that has at least 90% sequence identity to SEQ ID NO:37 and a heavy chain variable region comprising an amino acid sequence that has at least 90% sequence identity to SEQ ID NO:26.

19-32. (canceled)

33. A composition comprising an anti-MMP-9 antibody of claim 1, or fragment thereof, and a carrier.

34. (canceled)

35. An isolated polynucleotide comprising a nucleotide sequence encoding the isolated anti-MMP-9 antibody, or fragment thereof, of claim 1.

36. A vector comprising the polynucleotide of claim 35.

37. A cell comprising the polynucleotide of claim 35.

38. (canceled)

39. A method of detecting the presence of MMP-9 in a cell, the method comprising contacting the cell with an isolated anti-MMP-9 antibody of claim 1, or fragment thereof, and detecting whether a complex is formed between the anti-MMP-9 antibody and MMP-9.

40-41. (canceled)

42. A method of inhibiting the activity of MMP-9, comprising contacting MMP-9 with an isolated anti-MMP-9 antibody of claim 1, or fragment thereof.

43-46. (canceled)

47. A method for treating pain in a mammal, comprising administering an effective amount of an isolated anti-MMP-9 antibody of claim 1, or fragment thereof, to the mammal.

48-53. (canceled)

54. A method for treating a stroke in a mammal, comprising administering an effective amount of an isolated anti-MMP-9 antibody of claim 1, or fragment thereof, to the mammal.

55-58. (canceled)

59. A kit comprising an isolated anti-MMP-9 antibody of claim 1, or fragment thereof, packaging material, and instructions for administering the antibody, or a fragment thereof, to a mammal to treat pain or a stroke.

60-65. (canceled)


SEQUENCE LISTING (42 sequences)

SEQ ID NO: 1 - protein, 108 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSYHPLFTFGQGTKVEIKR

SEQ ID NO: 2 - protein, 11 aa, Artificial Sequence
  Description of Artificial Sequence: Synthetic peptide
RASQSVSSAVA

SEQ ID NO: 3 - protein, 7 aa, Artificial Sequence
  Description of Artificial Sequence: Synthetic peptide
SASSLYS

SEQ ID NO: 4 - protein, 9 aa, Artificial Sequence
  Description of Artificial Sequence: Synthetic peptide
QQSYHPLFT

SEQ ID NO: 5 - protein, 136 aa, Artificial Sequence
  Description of Artificial Sequence: Synthetic polypeptide
EVQLVESGGGLVQPGGSLRLSCAASGFNIYSYSIHWVRQAPGKGLEWVAYIYPSSGYTYYADSVKGRFTISADTSKNTAYLQMNSLRAEDTAVYYCARSSLAWAQDRVYKPVEAMTWAYGMDYWGQGTLVTVSSAS

SEQ ID NO: 6 - protein, 10 aa, Artificial Sequence
  Description of Artificial Sequence: Synthetic peptide
GFNIYSYSIH

SEQ ID NO: 7 - protein, 16 aa, Artificial Sequence
  Description of Artificial Sequence: Synthetic peptide
YIYPSSGYTYYADSVK

SEQ ID NO: 8 - protein, 25 aa, Artificial Sequence
  Description of Artificial Sequence: Synthetic peptide
SSLAWAQDRVYKPVEAMTWAYGMDY

SEQ ID NO: 9 - protein, 107 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQASHLITFGQGTKVEIKR

SEQ ID NO: 10 - skipped

SEQ ID NO: 11 - protein, 5 aa, Artificial Sequence
  Description of Artificial Sequence: Synthetic peptide
SRGSG

SEQ ID NO: 12 - protein, 8 aa, Artificial Sequence
  Description of Artificial Sequence: Synthetic peptide
QQASHLIT

SEQ ID NO: 13 - protein, 138 aa, Artificial Sequence
  Description of Artificial Sequence: Synthetic polypeptide
EVQLVESGGGLVQPGGSLRLSCAASGFNIYSSSMHWVRQAPGKGLEWVAYIYSSYGYTYYADSVKGRFTISADTSKNTAYLQMNSLRAEDTAVYYCARRFEPGLLKRNKRWISYTLCEAGYGMDYWGQGTLVTVSSAS

SEQ ID NO: 14 - skipped

SEQ ID NO: 15 - protein, 10 aa, Artificial Sequence
  Description of Artificial Sequence: Synthetic peptide
GFNIYSSSMH

SEQ ID NO: 16 - protein, 16 aa, Artificial Sequence
  Description of Artificial Sequence: Synthetic peptide
YIYSSYGYTYYADSVK

SEQ ID NO: 17 - protein, 27 aa, Artificial Sequence
  Description of Artificial Sequence: Synthetic peptide
RFEPGLLKRNKRWISYTLCEAGYGMDY

SEQ ID NO: 18 - protein, 107 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYAALITFGQGTKVEIKR

SEQ ID NO: 19 - skipped

SEQ ID NO: 20 - protein, 8 aa, Artificial Sequence
  Description of Artificial Sequence: Synthetic peptide
QQYAALIT

SEQ ID NO: 21 - protein, 138 aa, Artificial Sequence
  Description of Artificial Sequence: Synthetic polypeptide
EVQLVESGGGLVQPGGSLRLSCAASGFNISSSYIHWVRQAPGKGLEWVASISSSSGYTSYADSVKGRFTISADTSKNTAYLQMNSLRAEDTAVYYCARKYMVFGTRMGWVEHTDFAGQGYYAMDYWGQGTLVTVSSAS

SEQ ID NO: 22 - skipped

SEQ ID NO: 23 - protein, 10 aa, Artificial Sequence
  Description of Artificial Sequence: Synthetic peptide
GFNISSSYIH

SEQ ID NO: 24 - protein, 16 aa, Artificial Sequence
  Description of Artificial Sequence: Synthetic peptide
SISSSSGYTSYADSVK

SEQ ID NO: 25 - protein, 27 aa, Artificial Sequence
  Description of Artificial Sequence: Synthetic peptide
KYMVFGTRMGWVEHTDFAGQGYYAMDY

SEQ ID NO: 26 - protein, 138 aa, Artificial Sequence
  Description of Artificial Sequence: Synthetic polypeptide
EVQLVESGGGLVQPGGSLRLSCAASGFNIYSSSMHWVRQAPGKGLEWVASIYSYYGYTYYADSVKGRFTISADTSKNTAYLQMNSLRAEDTAVYYCARCKLYTSYMIPVGSDSVNRCMSSYGMDYWGQGTLVTVSSAS

SEQ ID NO: 27 - protein, 16 aa, Artificial Sequence
  Description of Artificial Sequence: Synthetic peptide
SIYSYYGYTYYADSVK

SEQ ID NO: 28 - protein, 27 aa, Artificial Sequence
  Description of Artificial Sequence: Synthetic peptide
CKLYTSYMIPVGSDSVNRCMSSYGMDY

SEQ ID NO: 29 - protein, 286 aa, Escherichia coli [sequence omitted]

SEQ ID NO: 30 - protein, 199 aa, Artificial Sequence
  Description of Artificial Sequence: Synthetic polypeptide
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGSRGSG

SEQ ID NO: 31 - protein, 6 aa, Artificial Sequence
  Description of Artificial Sequence: Synthetic peptide
RLPLGI

SEQ ID NO: 32 - protein, 10 aa, Artificial Sequence
  Description of Artificial Sequence: Synthetic peptide
SGRIGFLRTA

SEQ ID NO: 33-36 - skipped (4 entries)

SEQ ID NO: 37 - protein, 109 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQGGGASLITFGQGTKVEIKR

SEQ ID NO: 38 - protein, 10 aa, Artificial Sequence
  Description of Artificial Sequence: Synthetic peptide
QQGGGASLIT

SEQ ID NO: 39 - protein, 6 aa, Artificial Sequence
  Description of Artificial Sequence: Synthetic 6xHis tag
HHHHHH

SEQ ID NO: 40 - skipped

SEQ ID NO: 41 - protein, 5 aa, Artificial Sequence
  Description of Artificial Sequence: Synthetic peptide
SGGPW

SEQ ID NO: 42 - protein, 97 aa, Artificial Sequence
  Description of Artificial Sequence: Synthetic polypeptide
SGGPWELLTLASRQQLIDWMEADKVAGPLLRSALPAGWFIADKSGAGERGSRGIIAALGPDGKPSRIVVIYTTGSQATMDERNRQIAEIGASLIKHW